(19) United States
(12) Patent Application Publication
(10) Pub. No.: US 2006/0287402 A1
(43) Pub. Date: Dec. 21, 2006

(54) Cathepsin inhibitors

(76) Inventors: Christopher Bayly, Beaconsfield (CA); Cameron Black, Baic d'Urfe (CA); Daniel J. McKay, Ottawa (CA)

Correspondence Address: MERCK AND CO., INC, P O BOX 2000, RAHWAY, NJ, 07065-0907, US

(21) Appl. No.: 10/569,351

(22) PCT Filed: Aug. 23, 2004
(86) PCT No.: PCT/CA04/01577

§ 371 Date: Feb. 22, 2006

Related U.S. Application Data
(60) Provisional application No. 60/498,017, filed on Aug. 27, 2003.

(51) Int. Cl. A61K 31/13; C07C 211/01

(52) U.S. Cl. 514/663; 564/463

(57) ABSTRACT

This invention relates to a novel class of compounds, represented by the formula (I) below, wherein the meanings of R₁, R₂, R₃ and R₄ are indicated therein, which are cysteine protease inhibitors, including but not limited to, inhibitors of cathepsins K, L, S and B. These compounds are useful for treating diseases in which inhibition of bone resorption is indicated, such as osteoporosis, osteoarthritis and rheumatoid arthritis.

### FIELD OF THE INVENTION

The invention relates generally to inhibitors of protein activity, and specifically to cathepsin inhibitors.

### BACKGROUND OF THE INVENTION

Many cathepsins belong to the papain superfamily of cysteine proteases. These proteases function in the normal physiological as well as pathological degradation of connective tissue. Cathepsins play a major role in intracellular protein degradation and turnover and remodeling. These cathepsins are naturally found in a wide variety of tissues. To date, a number of cathepsin have been identified and sequenced from a number of sources; for example, cathepsin B, F, H, L, K, S, W, and Z have been cloned. Furthermore, the sequence of Cathepsin K can be found in PCT Application WO 96/13523, Khepri Pharmaceuticals, Inc., published May 9, 1996, which is hereby incorporated by reference in its entirety. Cathepsin L is implicated in normal lysosomal proteolysis as well as several diseases states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immunbe responses, including, but not limited to, rejection of organ transplants or tissue grafts. Increased Cathepsin B levels and redistribution of the enzyme are found in tumors, suggesting a role in tumor invasion and matastasis. In addition, aberrant Cathepsin B activity is implicated in such disease states as rheumatoid arthritis, osteoarthritis, pneumocystisis carinii, acute pancreatitis, inflammatory airway disease and bone and joint disorders.

Cysteine protease inhibitors such as E-64 (trans-epoxysuccinyl-L-leucylamide-(4-guanidino)butane) are known to be effective in inhibiting bone resorption. See Delaisse, J. M. et al., 1987, Bone 8:305-313, which is hereby incorporated by reference in its entirety. Recently, cathepsin K was cloned and found specifically expressed in osteoclasts See Tezuka, K. et al., 1994, J Biol Chem 269:1106-1109; Shi, G. P. et al., 1995, FEBS Lett 357:129-134; Bromme, D. and Okamoto, K., 1995, Biol Chem Hoppe Seyler 376:379-384; Bromme, D. et al., 1996, J Biol Chem 271:2126-2132; Drake, F. H. et al., 1996, J Biol Chem 271:12511-12516, which are hereby incorporated by reference in their entirety. Concurrent to the cloning, the autosomal recessive disorder, pycnodysostosis, characterized by an osteopetrotic phenotype with a decrease in bone resorption, was mapped to mutations present in the cathepsin K gene. To date, all mutations identified in the cathepsin K gene are known to result in inactive protein. See Gelb, B. D. et al., 1996, Science 273:1236-1238; Johnson, M. R. et al., 1996, Genome Res 6:1050-1055, which are hereby incorporated by reference in their entirety. Therefore, it appears that cathepsin K is involved in osteoclast mediated bone resorption.

Cathepsin K is synthesized as a 37 kDa pre-pro enzyme, which is localized to the lysosomal compartment and where it is presumably autoactivated to the mature 27 kDa enzyme at low pH. See McQueney, M. S. et al., 1997, J Biol Chem 272:13955-13960; Littlewood-Evans, A. et al., 1997, Bone 20:81-86, which are hereby incorporated by reference in their entirety. Cathepsin K is most closely related to cathepsin S having 56% sequence identity at the amino acid level. The S₂P₂ substrate specificity of cathepsin K is similar to that of cathepsin S with a preference in the P1 and P2 positions for a positively charged residue such as arginine, and a hydrophobic residue such as phenylalanine or leucine, respectively. See Bromme, D. et al., 1996, J Biol Chem 271: 2126-2132; Bossard, M. J. et al., 1996, J Biol Chem 271:12517-12524, which are hereby incorporated by reference in their entirety. Cathepsin K is active at a broad pH range with significant activity between pH 4-8, thus allowing for good catalytic activity in the resorption lacunae of osteoclasts where the pH is about 4-5.

Human type I collagen, the major collagen in bone is a good substrate for cathepsin K. See Kafienah, W., et al., 1998, Biochem J 331:727-732, which is hereby incorporated by reference in its entirety. In vitro experiments using antisense oligonucleotides to cathepsin K, have shown diminished bone resorption in vitro, which is probably due to a reduction in translation of cathepsin K mRNA. See Inui, T., et al., 1997, J Biol Chem 272:8109-8112, which is hereby incorporated by reference in its entirety. The crystal structure of cathepsin K has been resolved. See McGrath, M. E., et al., 1997, Nat Struct Biol 4:105-109; Zhao, B., et al., 1997, Nat Struct Biol 4: 109-11, which are hereby incorporated by reference in their entirety. Also, selective peptide based inhibitors of cathepsin K have been developed See Bromme, D., et al., 1996, Biochem J 315:85-89; Thompson, S. K., et al., 1997, Proc Natl Acad Sci USA 94:14249-14254, which are hereby incorporated by reference in their entirety. Accordingly, inhibitors of Cathepsin K can reduce bone resorption. Such inhibitors would be useful in treating disorders involving bone resorption, such as osteoporosis.

### SUMMARY OF THE INVENTION

The invention provides compounds that are capable of treating and/or preventing cathepsin dependent conditions or disease states in a mammal in need thereof. The invention provides compounds of the generic formula: 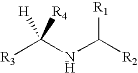
where R₄ is a non-hydrogen substituent with electron-withdrawing character adequate to, in conjunction with R₁, R₂ and R₃, reduce the pKa of the nitrogen to <6 in aqueous media, where R₁ is a substituent binding in the S₁ subsite of the active site of cathepsins, and where R₂ is a substituent binding in the S₂ subsite of the active site of cathepsins, and where R₃ is a substituent binding in the S₃ subsite of the active site of cathepsins.

More specifically, the invention provides compounds of the formula: 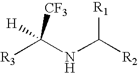
where R₁ is a substituent binding in the S₁ subsite of the active site of cathepsins, and where R₂ is a substituent binding in the S₂ subsite of the active site of cathepsins, and where R₃ is a substituent binding in the S₃ subsite of the active site of cathepsins.

A special nomenclature has evolved for describing the active site of a protease inhibitor. See, U.S. Pat. No. 6,333,402, incorporated herein by reference. Starting at the residue on the amino side of the scissile bond of the substrate, and moving away from the bond, residues are named P₁, P₂, P₃, etc. Residues that follow the scissile bond are called P₁′, P₂′, P₃′, etc. It has been found that the main chain of protein inhibitors having very different overall structure are highly similar in the region between P₃ and P₃′ with especially high similarity for P₂, P₁ and P₁′. It is generally accepted that each protease active site has subsites S₁, S₂, etc. that receive the side groups of residues P₁, P₂, etc. of the substrate or inhibitor and subsites S₁′, S₂′, etc. that receive the side groups of P₁′, P₂′, etc. of the substrate or inhibitor. It is the interactions between the S subsites and the P side groups that give the protease specificity with respect to substrates and the inhibitors specificity with respect to proteases. This nomenclature is generalized to refer to non-peptide inhibitors of proteases where the region of the non-peptide inhibitor that interacts with protease subsites S₁, S₂, etc., would be referred to as P₁, P₂, etc., respectively.

The P₂-P₃ amide bond in dipeptidic cathepsin inhibitors may be replaced by a substituted ethylamine moeity. The electron-withdrawing properties of the R₁, R₂, R₃, and R₄ groups together render the P2 amine non-basic at physiological pH. This fragment is thus capable of making an important neutral hydrogen bond to a cathepsin glycine conserved amongst the entire papain family of cathepsins.

Both amides and anilines are capable of making hydrogen bonds to carbonyl groups, but have metabolic liabilities. By itself, the ethylamine is basic and is protonated in biological systems, generating a charge that reduces binding to the target enzyme. With sufficiently electron-withdrawing R₄ (or more specifically trifluoroethylamine), the amine is not protonated and provides a superior hydrogen bond to acceptor oxygen in the cathepsin active site. In cathepsins, an important acceptor oxygen is situated in between the S₂ and S₃ subsites of the active site; in cathepsin K this is Gly66. This residue is a conserved residue amongst this entire papain family of proteins, including cathepsins B, F, H, K, L, L2, O, S, W, and Z, falcipain, falcipain-1 and falcipain 2. Thus, using the non-basic ethylamine linker to join inhibitor fragments binding the S₂ and S₃ subsites of cathepsins allows for more potent inhibition than the corresponding amides.

### DETAILED DESCRIPTION OF THE INVENTION

The present invention relates to a composition of matter having the chemical formula: 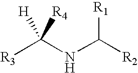
and having no more than 70 nonhydrogen atoms each independently selected from C, O, N, S, P, F, Cl, Br or I;
wherein R₄ is a non-hydrogen electron-withdrawing substituent such that, together with R₁, R₂ and R₃, the basicity of the nitrogen is lowered to less than a pKa of 6;
wherein a molecule of the composition is interacting with a cathepsin such that the CH—NH region in the chemical formula is interacting favorably with the cathepsin between S₂ and S₃, R₁ interacts favorably with S₁ but not S₃ of the cathepsin active site, R₂ interacts favorably with S₂ but not S₃ of the cathepsin active site, and R₃ interacts favorably with S₃ but not S₂ or S₁ of the cathepsin active site.

In a class of the invention the cathepsin is selected from cathepsin B, P, H, K, L, L2, O, S, W and Z. In a subclass of the invention, the cathepsin is selected from cathepsin K, L, S, and B. In a further subclass of the invention, the cathepsin is cathepsin L. In a further subclass of the invention, the cathepsin is cathepsin S. In a further subclass of the invention, the cathepsin is cathepsin B. In a further subclass of the invention, the cathepsin is cathepsin F.

In a class of the invention, R₄ does not interact favorably with subsites S₂, S₃ and S₁, respectively, of a cathepsin active site. In another class of the invention, R₄ is selected from —CF₃, —CHF₂, —CH₂F, —CF₂R₅, and —CHF R₅, wherein R₅ is C₁₋₆ alkyl, aryl, or heteroaryl optionally substituted with 1 to 4 substituents selected from halo, C₁₋₃ alkyl, C₁₋₃ alkoxy, hydroxy, hydroxyalkyl, keto, cyano, heterocyclyl, C₃₋₈ cycloalkyl, SOₘC₁₋₃ alkyl, NH₂, NO₂ or O(C═O)C₁₋₃ alkyl, wherein m is an integer from zero to two.

In a class of the invention, R₂ has at least one carbon or sulfur atom which simultaneously fulfills the following three distance critieria: it is within 7 Å of Cα₂₆, and it is within 8.5 Å of Cα₆₈ and it is within 7 Å of Cα₁₃₄ of a cathepsin. In another class of the invention, R₂ comprises nonpolar regions. In another class of the invention, R₂ comprises lipophilic regions.

In a class of the invention, R₁ comprises a region that stably fits into subsite S₁ of a cathepsin active site, having at least one carbon atom within 5 Å of Cα₂₅ of a cathepsin. In another class of the invention, R₁ is non-immunogenic

In a class of the invention, R₃ has at least one carbon or sulfur atom which simultaneously fulfills the following two distance critieria: it is within 5.5 Å of Cα₆₆, and it is within 7 Å of Cα₆₀ of a cathepsin. In another class of the invention, R₃ comprises nonpolar regions. In another class of the invention, R₃ comprises lipophilic regions.

In a class of the invention, the nitrogen has a pKa of less than 6 and makes a hydrogen bond with the cathepsin amide carbonyl of glycine 66 of a cathepsin.

In a class of the invention, the compound has a molecular weight of less than 1000 daltons. In a class of the invention, the compound forms a covalent bond with cysteine 25 of a cathepsin. In a class of the invention, the compound binds to the active site of a cathepsin with an IC₅₀ of less than 10 micromolar in a purified enzyme assay.

In a class of the invention, the pKa of the nitrogen of the secondary amine shown in claim 1 is <5 in an aqueous medium.

The term “lipophilic,” as the term is used herein, refers to a compound, which, as a separate entity, is more soluble in nonpolar solvents (e.g. cyclohexane) than water. The term “lipophilic group”, in the context of being attached to a molecule, refers to a region of the molecule having high hydrocarbon content thereby giving the group high affinity to nonpolar solvents or lipid phases. A lipophilic group can be, for example, an alkyl or cycloalkyl chain(preferably n-alkyl) of less than 30 carbons. To further illustrate, lipophilic groups include the alkyl chains attached to naturally-occurring and synthetic aromatic and non-aromatic moieties such as fatty acids, esters and alcohols, and other lipid molecules. Other examples of lipophilic molecules are cage structures such as adamantane and buckminsterfullerenes, and aromatic hydrocarbons such as benzene, perylene, phenanthrene, anthracene, naphthalene, pyrene, chrysene, and naphthacene. Specifically included in the term “lipophilic group” as used herein are the carbons and attached hydrogens of alkyl chains, cycloalkyl chains, aryl rings, or heteroaryl rings. The term “lipophilic group” as used herein also includes divalent sulfur.

The term “substantially homologous”, when used in connection with amino acid sequences, refers to sequences which are substantially identical to or similar in sequence, giving rise to a homology in conformation and thus to similar biological activity. The term is not intended to imply a common evolution of the sequences. Typically, “substantially homologous” sequences are at least 50% more preferably at least 80% identical in sequence, at least over any regions known to be involved in the desired activity. Most preferably, no more than five residues, other than at the termini, are different. Preferably, the divergence in sequence, at least in the aforementioned regions, is in the form of “conservative modifications”.

“Conservative modifications” are defined as (a) conservative substitutions of amino acids as hereafter defined; and (b) single or multiple insertions or deletions of amino acids at the termini, at interdomain boundaries, in loops or in other segments of relatively high mobility (as indicated, e.g., by the failure to clearly resolve their structure upon X-ray diffraction analysis or NMR). Preferably, except at the termini, no more than about five amino acids are inserted or deleted at a particular locus, and the modifications are outside regions known to contain binding sites important to activity.

Conservative substitutions are herein defined as exchanges within one of the following five groups: (1) small aliphatic, nonpolar or slightly polar residues: Ala, Ser, Thr (Pro, Gly); (2) polar, negatively charged residues: and their amides Asp, Asn, Glu, Gln; (3) polar, positively charged residues: His, Arg, Lys; (4) large, aliphatic, nonpolar residues: Met, Leu, Ile, Val (Cys); and (5) large, aromatic residues: Phe, Tyr, Trp. Residues Pro, Gly and Cys are parenthesized because they have special conformational roles. Cys participates in formation of disulfide bonds. Gly imparts flexibility to the chain. Pro imparts rigidity to the chain and disrupts alpha helices. These residues may be essential in certain regions of the polypeptide, but substitutable elsewhere. Semi-conservative substitutions are defined to be exchanges between two of groups (1)-(5) above which are limited to supergroup (a), comprising (1), (2) and (3) above, or to supergroup (b), comprising (4) and (5) above.

The term “cathepsin” or “cathepsins” as used herein refers to enzymes belonging to the papain family, i.e. where the active form of the enzyme is folded similarly to papain (Conserved Domain smart00645.7 in the NCBI Conserved Domain Database http: //www.ncbi.nlm.nih.gov/Structure/cdd/cddsrv.cgi?uid=15045). Only the cathepsins in human, mouse, rabbit, primates, rat, and plasmodium falciparum are being referred to herein. The human cathepsins specifically included are B, F, H, K, L, L2, O, S, W, and Z; also specifically included are enzymes in mouse, rabbit, primates, and rat exhibiting greater than 80% sequence identity (comparing the active forms of the enzymes) to the most similar of the preceding human cathepsins. Also specifically included are falcipain, falcipain-1 and falcipain-2 from plasmodium falciparum, and any other enzymes from plasniodiumfalciparum exhibiting greater than 80% sequence identity to the most similar of these, again comparing the active forms of the enzymes.

A specific amino acid residue(e.g. glycine 66) or a Ca (e.g. Cα₆₆) in a cathepsin is referred to herein via a combination of the residue numbering used for the active form of Cathepsin K and the primary sequence alignment given in FIG. 1. This sequence alignment is for the relevant portion of the active form of the protein for human cathepsins and falcipains as labeled in the Figure. In the Figure, some of the key residue numbers referred to herein are given, formatted vertically above the aligned sequences. Other residue numbers referred to can found by counting along from the closest numbered residue or by direct reference to the Cathepsin K sequence. The numbering is obtained based on the SWISSPROT primary accession #P43235 for human cathepsin K, where the first residue in the CHAIN, the active form of the protein (residue number 115 in #P43235) is reset herein to be residue number 1. The sequence alignment herein is used to generalize the reference to a Cathepsin K residue to the other human cathepsins and the falcipains. The reference to residues in mouse, rabbit, primates, and rat cathepsins is implied by a standard primary sequence alignment, using default parameters, to the most highly homologous sequence specified below:

| | |
|---|---|
| 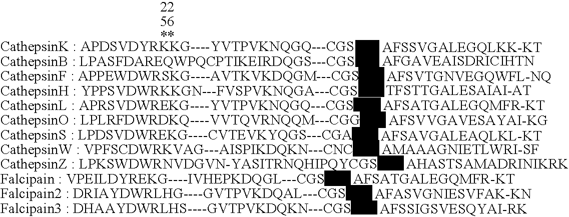 | FIG. 1. |
| 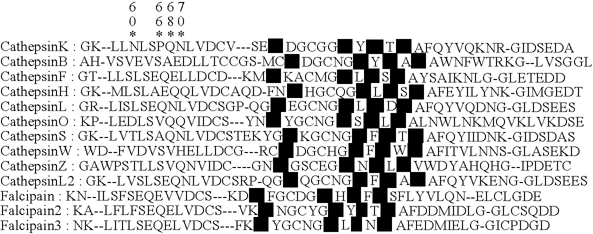 | |
| 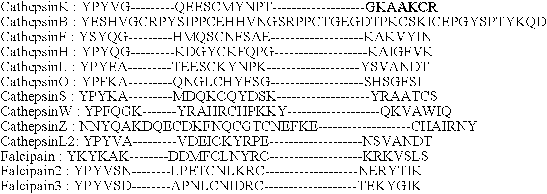 | |
| 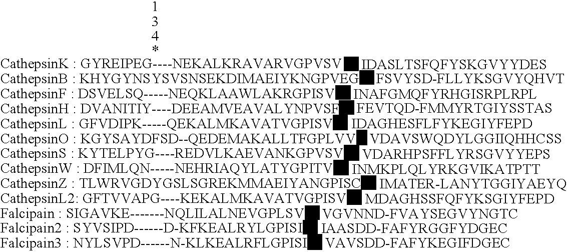 | |
| 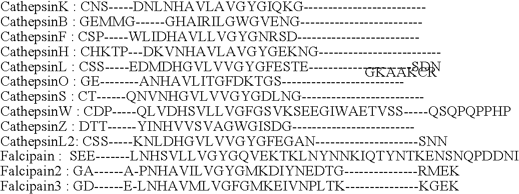 | |
| 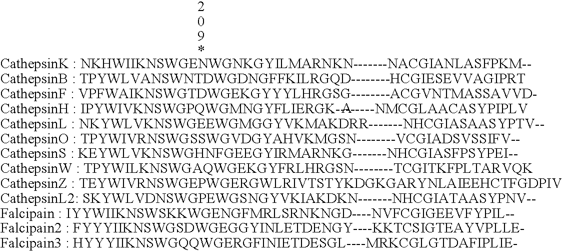 | |
| 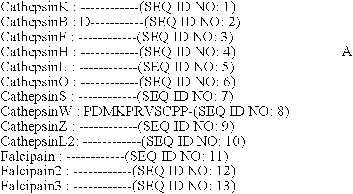 | |

Definitions: The term “favorably”, as used herein in the term “interacts favorably”, refers to the favorable result of a molecular modeling calculation in which a computer model of the ligand molecule is placed into the active site of a computer model of the cathepsin, making a ligand:cathepsin complex, and the ligand:cathepsin complex is energy minimized using a standard molecular mechanics forcefield such as MME94s (T. A. Halgren, Journal of Computatioinal Chemistry (1999), 20, pp. 720-729.). The initial placement of the ligand into the active site, also referred to as “docking”, is done so as to optimally allow the ligand:enzyme interactions between the substituents of the ligand and the subsites of the enzyme as stated herein. The computer model of the active site is based on an Xray crystal structure (e.g. Protein Databank entry 1MEM for cathepsin K) if available for the cathepsin of interest (minus the non-protein atoms), or if an Xray crystal structure is not available, one can use a homology-built structure based on the most similar cathepsin for which an Xray structure is available, or simply use directly the most similar cathepsin for which an Xray structure is available. At the start of the energy minimization, no protein atoms have been adjusted to accommodate the ligand, but during the energy minimization, entire protein sidechains having at least one atom within 6 Å of the ligand are allowed to move with the energy minimization. No mainchain atoms of the enzyme are allowed to move during the energy minimization. A continuum dielectric solvent of water may be used, but otherwise default parameters and behaviour of a standard molecular mechanics energy minimization similar to those in Schrodinger Inc.'s software program MacroModel is assumed . A “favorable result of a modeling calculation” means that after the energy minimization is complete there are no bad steric interactions between the ligand and the active site and there are energy-stabilising hydrogen bonding and lipophilic interactions between the ligand and the active site. If it is believed that the ligand forms a covalent bond with the active site sulfur of cysteine-25, the energy minimization may include that covalent bond in the modeled ligand:cathepsin complex submitted to the calculation. Interactions between the ligand and the cathepsin as claimed herein are based on the geometry of the ligand:cathepsin complex that would result from such an energy minimization.

In one embodiment of the invention, favorable interactions between a substituent of the ligand and S₂ require having at least one carbon or divalent sulfur atom of the substituent simultaneously fulfilling the following three distances: it is within 7 Å of Cα₂₆, and it is within 8.5 Å of Cα₆₈ and it is within 7 Å of Cα₁₃₄ of the cathepsin. In another embodiment of the invention, favorable interactions between a substituent of the ligand and S₂ require having at least one atom of a lipophilic group of the substituent within 5.5 Å of atoms of two of residues 67, 68, 134. In a further embodiment of the invention relating only to cathepsin B, favorable interactions between a substituent of the ligand and S₂ require having hydrogen bonding between the substituent and glutamate 209 of cathepsin B.

In one embodiment of the invention, favorable interactions between a substituent of the ligand and S₃ require having at least one carbon or divalent sulfur atom of the substituent simultaneously fulfilling the following two distances: it is within 5.5 Å of Cα₆₆, and it is within 7 Å of Cα₆₀ of the cathepsin. In another embodiment of the invention, favorable interactions between a substituent of the ligand and S₃ require having at least one atom of a lipophilic group of the substituent within 5.5 Å of Cα₆₆ and atoms of either residue 60 or 61. In another embodiment of the invention, favorable interactions between a substituent of the ligand and S₃ require having at least one atom of a lipophilic group of the substituent within 5.5 Å of either residue 60 or 61.

In one embodiment of the invention, favorable interactions between a substituent of the ligand and S₁ require having at least one carbon atom of the substituent within 5 Å of Cα₂₅. In another embodiment of the invention, favorable interactions between a substituent of the ligand and S₁ require having a covalent bond between the active site cysteine sulfur of residue 25 and an electrophilic carbon of the ligand, preferably a carbonyl or nitrile carbon.

In one embodiment of the invention, favorable interactions between the CH—NH region in the chemical formula of the ligand and the cathepsin between S2 and S3 requires having the N of the chemical formula within 4 Å of the peptide oxygen of Gly66, and the C of the chemical formula (referring only the C directly connected with R₄) within 5.5 Å of Cα₆₆. In another embodiment of the invention, favorable interactions between the CH—NH region in the chemical formula of the ligand and the cathepsin between S2 and S3 requires having the NH of the chemical formula hydrogen bonding to the amide O of residue 66.

As used herein, “alkyl” is intended to include both branched and straight-chain saturated aliphatic hydrocarbon groups having one to ten carbon atoms unless otherwise specified. For example, C₁-C₁₀, as in “C₁-C₁₀ alkyl” is defined to include groups having 1, 2, 3, 4, 5, 6, 7, 8, 9 or 10 carbons in a linear, branched, or cyclic arrangement. For example, “C₁-C₁₀ alkyl” specifically includes methyl, ethyl, propyl, butyl, pentyl, hexyl, heptyl, octyl, nonyl, decyl, and so on.

“Alkoxy” or “alkyloxy” represents an alkyl group as defined above, unless otherwise indicated, wherein said alkyl group is attached through an oxygen bridge.

The term “cycloalkyl” or “carbocycle” shall mean cyclic rings of alkanes of three to eight total carbon atoms, unless otherwise indicated, or any number within this range (i.e., cyclopropyl, cyclobutyl, cyclopentyl, cyclohexyl, cycloheptyl or cyclooctyl).

As used herein, “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or tricyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyridazinyl, pyridopyridinyl, pyridyl, pyrimnidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, methylenedioxybenzene, benzothiazolyl, benzothienyl, quinolinyl, isoquinolinyl, oxazolyl, and tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo” or “halogen” as used herein is intended to include chloro, fluoro, bromo and iodo. The term “keto” means carbonyl (C═O). The term “alkoxy” as used herein means an alkyl portion, where alkyl is as defined above, connected to the remainder of the molecule via an oxygen atom. Examples of alkoxy include methoxy, ethoxy and the like.

The term “hydroxyalkyl” means a linear monovalent hydrocarbon raidcal of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, and the like.

The term “heterocycle” or “heterocyclyl” as used herein is intended to mean a 5- to 10-membered nonaromatic ring, unless otherwise specified, containing from 1 to 4 heteroatoms selected from the group consisting of O, N, S, SO, or SO₂ and includes bicyclic groups. “Heterocyclyl” therefore includes, but is not limited to the following: piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also emcompassed by this definition.

The present invention also includes N-oxide derivatives and protected derivatives of compounds of Formula I. For example, when compounds of Formula I contain an oxidizable nitrogen atom, the nitrogen atom can beconverted to an N-oxide by methods well known in the art. Also whencompounds of Formula I contain groups such as hydroxy, carboxy, thiol or anygroup containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

Also included within the scope of the present invention is a pharmaceutical composition which is comprised of a compound as described above and a pharmaceutically acceptable carrier. The invention is also contemplated to encompass a pharmaceutical composition, which is comprised of a pharmaceutically acceptable carrier and any of the compounds specifically disclosed in the present application. These and other aspects of the invention will be apparent from the teachings contained herein.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci., 1977:66: 1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

The compounds of the present invention are inhibitors of cathepsins and are therefore useful to treat or prevent cathepsin dependent diseases or conditions in mammals, preferably humans. Specifically, the compounds of the present invention are inhibitors of Cathepsin K and are therefore useful to treat or prevent Cathepsin K dependent diseases or conditions in mammals, preferably humans.

“Cathepsin dependent diseases or conditions” refers to pathologic conditions that depend on the activity of one or more cathepsins. “Cathepsin K dependent diseases or conditions” refers to pathologic conditions that depend on the activity of Cathepsin K. Diseases associated with Cathepsin K activities include osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, obesity, atherosclerosis, chronic obstructive pulmonary disease, juvenile onset diabetes, multiple sclerosis, pemphigus vulgaris, Graves' disease, myasthenia gravis, systemic lupus erythemotasus, rheumatoid arthritis and Hashimoto's thyroiditis, asthma, allogenic immune responses, parasitic infection, cancer, metastatic bone disease, hypercalcemia of malignancy or multiple myeloma. In treating such conditions with the instantly claimed compounds, the required therapeutic amount will vary according to the specific disease and is readily ascertainable by those skilled in the art. Although both treatment and prevention are contemplated by the scope of the invention, the treatment of these conditions is the preferred use.

An embodiment of the invention is a method of inhibiting cathepsin activity in a mammal in need thereof, comprising administering to the mammal a therapeutically effective amount of any of the compounds or any of the pharmaceutical compositions described above.

A class of the embodiment is the method wherein the cathepsin activity is cathepsin K activity.

Another embodiment of the invention is a method of treating or preventing cathepsin dependent conditions in a mammal in need thereof, comprising administering to the mammal a therapeutically effective amount of any of the compounds or any of the pharmaceutical compositions described above.

A class of the embodiment is the method wherein the cathepsin activity is cathepsin K activity.

Another embodiment of the invention is a method of inhibiting bone loss in a mammal in need thereof, comprising administering to the mammal a therapeutically effective amount of any of the compounds or any of the pharmaceutical compositions described above. Another embodiment of the invention is a method of reducing bone loss in a mammal in need thereof, comprising administering to the mammal a therapeutically effective amount of any of the compounds or any of the pharmaceutical compositions described above. The utility of cathepsin K inhibitors in the inhibition of bone resorption is known in the literature. See Stroup G. B. et al., “Potent and selective inhibition of human cathepsin K leads to inhibition of bone resorption in vivo in a nonhuman primate.” J. Bone Miner. Res., 16:1739-1746;2001; and Votta, B. J. et al., “Peptide aldehyde inhibitors of cathepsin K inhibit bone resorption both in vivo and in vitro.” J. Bone Miner. Res. 12:1396-1406; 1997.

Another embodiment of the invention is a method of treating or preventing osteoporosis in a mammal in need thereof, comprising administering to the mammal a therapeutically effective amount of any of the compounds or any of the above pharmaceutical compositions described above. The utility of cathepsin K inhibitors in the treatment or prevention of osteoporosis is known in the literature. See, Saftig P. et al., “Impaired osteoclast bone resorption leads to osteoporosis in cathepsin K-deficient mice.” Proc. Natl. Acad. Sci. USA 95:13453-13458; 1998.

Another embodiment of the invention is a method of treating or preventing rheumatoid arthritic condition in a mammal in need thereof, comprising administering to the mammal a therapeutically effective amount of any of the compounds or any of the pharmaceutical compositions described above. It is known in the literature that progressive destruction of the periarticular bone is a major cause of joint dysfunction and disability in patients with rheumatoid arthritis (RA). See Goldring S R, “Pathogenesis of bone erosions in rheumatoid arthritis”. Curr. Opin. Rheumatol. 14: 406-10, 2002. Analysis of joint tissues from patients with RA have provided evidence that cathepsin K positive osteoclasts are the cell types that mediate the focal bone resorption associated with rheumatoid synovial lesion. See Hou, W-S et al., “Comparision of Cathepsin K and S expression within the Rheumatoid and Osteoarthritic Synovium”, Arthritis Rheumatism 46: 663-74, 2002. In addition, generalized bone loss is a major cause of morbility associated with severe RA. The frequency of hip and spinal fractures is substantially increased in patients with chronic RA. See, Gould et al, “Osteoclastic activation is the principal mechanism leading to secondary osteoporosis in rheumatoid arthritis”. J. Rheumatol. 25: 1282-9, 1998. The utility of cathepsin K inhibitors in the treatment or prevention of resorption in subarticular bone and of generalized bone loss represent a rational approach for pharmacological intervention on the progression of rheumatoid arthritis.

Another embodiment of the invention is a method of treating or preventing the progression of osteoarthritis in a mammal in need thereof, comprising administering to the mammal a therapeutically effective amount of any of the compounds or any of the pharmaceutical compositions described above. It is known in the literature that osteoarthritis (OA) is accompanied with a well-defined changes in the joints, including erosion of the articular cartilage surface, peri-articular endochondral ossification/osteophytosis, and subchondral bony sclerosis and cyst formation, see Oettmeier R & Abendroth, K, “Osteoarthritis and bone: osteologic types of osteoarthritis of the hip”, Skeletal Radiol. 18: 165-74, 1989. Recently, the potential contribution of subchondral bone sclerosis to the initiation and progression of OA have been suggested. Stiffened subchondral bone as the joint responding to repetitive impulsive loading, is less able to attenuate and distribute forces through the joint, subjecting it to greater mechanical stress across the articular cartilage surface. This in turn accelerates cartilage wear and fibrillate. See, Radin, E L and Rose R M, “Role of subchondral bone in the initiation and progression of cartilage damage.” Clin. Orthop. 213: 34-40, 1986. Inhibition of excessive subarticular bone resorption by an anti-resorptive agent such as a cathepsin K inhibitor, will lead to inhibition of subchondral bone turnover, thus may have a favorable impact on OA progression. In addition to the above hypothesis, cathepsin K protein expression was recently identified in synovial fibroblasts, macrophage-like cells, and chondrocytes from synovium and articular cartilage specimens derived from OA patients. See, Hou, W-S et al., “Comparison of Cathepsin K and S expression within the Rheumatoid and Osteoarritic Synovium”, Arthritis Rheumatism 46: 663-74, 2002; and Dodd RA et al., “Expression of Cathepsin K messenger RNA in giant cells and their precursors in human osteoarthritic synovial tissues”. Arthritis Rheumatism 42: 1588-93, 1999; and Konttinen et al., “Acidic cysteine endoproteinase cathepsin K in the degeneration of the superficial articular hyaline cartilage in osteoarthritis”, Arthritis Rheumatism 46: 953-60, 2002. These recent studies thus implicated the role of cathepsin K in the destruction of collagen type II in the articular cartilage associated with the progression of osteoarthritis. The utility of cathepsin K inhibitors in the treatment or prevention of osteoarthritis as described in this invention thus comprise of two different mechanisms, one is on the inhibition of osteoclast-driven subchondral bone turnover, and two is on the direct inhibition of collagen type II degeneration in the synovium and cartilage of patients with OA.

Another embodiment of the invention is a method treating cancer in a mammal in need thereof, comprising administering to the mammal a therapeutically effective amount of any of the compounds or any of the pharmaceutical compositions described above. It is known in the literature that cathepsin K is expressed in human breast carcinoma. See Littlewood-Evans A J et al., “The osteoclast-associated protease cathepsin K is expressed in human breast carcinoma.” Cancer Res 57(23):5386-90, Dec. 1, 1997.

Exemplifying the invention is the use of any of the compounds described above in the preparation of a medicament for the treatment and/or prevention of osteoporosis in a mammal in need thereof. Still further exemplifying the invention is the use of any of the compounds described above in the preparation of a medicament for the treatment and/or prevention of: bone loss, bone resorption, bone fractures, metastatic bone disease and/or disorders related to cathepsin functioning.

The compounds of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

In the case of tablets for oral use, carriers which are commonly used include lactose and corn starch, and lubricating agents, such as magnesium stearate, are commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The compounds of the present invention can also be administered in the form of liposome delivery systems, such as small unilamellar vesicles, large unilamellar vesicles and multilameflar vesicles. Liposomes can be formed from a variety of phospholipids, such as cholesterol, stearylamine or phosphatidylcholines.

Compounds of the invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palnitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The instant compounds are also useful in combination with known agents useful for treating or preventing osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, obesity, atherosclerosis, chronic obstructive pulmonary disease, juvenile onset diabetes, multiple sclerosis, pemphigus vulgaris, Graves' disease, myasthenia gravis, systemic lupus erythemotasus, rheumatoid arthritis and Hashimoto's thyroiditis, asthma, allogenic immune responses, parasitic infection, cancer, metastatic bone disease, hypercalcemia of malignancy or multiple myeloma. Combinations of the presently disclosed compounds with other agents useful in treating or preventing osteoporosis or other bone disorders are within the scope of the invention. A person of ordinary skill in the art would be able to discern which combinations of agents would be useful based on the particular characteristics of the drugs and the disease involved. Such agents include the following: an organic bisphosphonate; an estrogen receptor modulator; an androgen receptor modulator; an inhibitor of osteoclast proton ATPase; an inhibitor of HMG-CoA reductase; an integrin receptor antagonist; an osteoblast anabolic agent, such as PmH; and the pharmaceutically acceptable salts and mixtures thereof. A preferred combination is a compound of the present invention and an organic bisphosphonate. Another preferred combination is a compound of the present invention and an estrogen receptor modulator. Another preferred combination is a compound of the present invention and an androgen receptor modulator. Another preferred combination is a compound of the present invention and an osteoblast anabolic agent.

“Organic bisphosphonate” includes, but is not limited to, compounds of the chemical formula 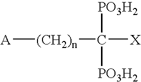
wherein n is an integer from 0 to 7 and wherein A and X are independently selected from the group consisting of H, OH, halogen, NH₂, SH, phenyl, C1-C30 alkyl, C3-C30 branched or cycloalkyl, bicyclic ring structure containing two or three N, C1-C30 substituted alkyl, C1-C10 alkyl substituted NH₂, C3-C10 branched or cycloalkyl substituted NH₂, C1-C10 dialkyl substituted NH₂, C1-C1alkoxy, C1-C10 alkyl substituted thio, thiophenyl, halophenylthio, C1-C10 alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C3-C10 ring.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH₂, C1-C10 alkyl or dialkyl substituted NH₂, OH, SH, and C1-C10 alkoxy.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C1-C30-alkyl-substituted ammonium. Preferred salts are those selected from the group consisting of sodium, potassium, calcium, magnesium, and ammonium salts. More preferred are sodium salts. Non-limiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein. For example, the phrase “about 5 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 5 mg of alendronic acid.

Non-limiting examples of bisphosphonates useful herein include the following: Alendronate, also known as alendronic acid, alendronate sodium, alendronate monosodium trihydrate or 4-amino-1-hydroxybutylidene-1,1-bisphosphonic acid monosodium trihydrate. Alendronate is described in U.S. Pat. No. 4,922,007, to Kieczykowski et al., issued May 1, 1990; U.S. Pat. No. 5,019,651, to Kieczykowski et al., issued May 28, 1991; U.S. Pat. No. 5,510,517, to Dauer et al., issued Apr. 23, 1996; U.S. Pat. No. 5,648,491, to Dauer et al., issued Jul. 15, 1997, all of which are incorporated by reference herein in their entirety.
Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.
1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.
1-hydroxy-3-(1-pyrrolidinyl)-propylidene-1,1-bisphosphonic acid (EB-1053).
1-hydroxyethane-1,1-diphosphonic acid (etidronic acid).
1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.
1-hydroxy-2-imidazo-(1,2-a)pyridin-3-yethylidene (minodronate).
6-amino-1-hydroxyhexylidene-1,1-bisphosphonic acid (neridronate).
3-(dimethylamino)-1-hydroxypropylidene-1,1-bisphosphonic acid (olpadronate).
3-amino-1-hydroxypropylidene-1,1-bisphosphonic acid (pamidronate).
[2-(2-pyridinyl)ethylidene]-1,1-bisphosphonic acid (piridronate) is described in U.S. Pat. No. 4,761,406, which is incorporated by reference in its entirety.
1-hydroxy-2-(3-pyridinyl)-ethylidene-1,1-bisphosphonic acid (risedronate).
(4-chlorophenyl)thiomethane-1,1-disphosphonic acid (tiludronate) as described in U.S. Pat. No. 4,876,248, which is incorporated by reference herein in its entirety.
1-hydroxy-2-(1H-imidazol-1-yl)ethylidene-1,1-bisphosphonic acid (zoledronate).

Nonlimiting examples of bisphosphonates include alendronate, cimadronate, clodronate, etidronate, ibandronate, incadronate, minodronate, neridronate, olpadronate, paamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

It is recognized that mixtures of two or more of the bisphosphonate actives can be utilized.

The precise dosage of the organic bisphosphonate will vary with the dosing schedule, the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight. For alendronate monosodium trihydrate, common human doses which are administered are generally in the range of about 2 mg/day to about 40 mg/day, preferably about 5 mg/day to about 40 mg/day. In the U.S. presently approved dosages for alendronate monosodium trihydrate are 5 mg/day for preventing osteoporosis, 10 mg/day for treating osteoporosis, and 40 mg/day for treating Paget's disease.

In alternative dosing regimens, the bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of 35 mg/week or 70 mg/week.

“Selective estrogen receptor modulators” refers to compounds which interfere or inhibit the binding of estrogen to the receptor, regardless of mechanism. Examples of estrogen receptor modulators include, but are not limited to, estrogen, progestogen, estradiol, droloxifene, raloxifene, lasofoxifene, TSE-424, tamoxifen, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-2,2-dimethylpropanoate, 4,4′-dihydroxybenzophenone-2,4-dinitrophenyl-hydrazone, and SH646.

An “estrogen receptor beta modulator” is a compound that selectively agonizes or antagonizes estrogen receptor beta (ER [symbol] Agonizing ER□ increases transcription of the tryptophan hydroxylase gene (TPH, the key enzyme in serotonin synthesis) via an ER□ mediated event. Examples of estrogen receptor beta agonists can be found in PCI International publication WO 01/82923, which published on Nov. 08, 2001, and WO 02/41835, which published on May 20, 2002, both of which are hereby incorporated by reference in their entirety.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“An inhibitor of osteoclast proton ATPase” refers to an inhibitor of the proton ATPase, which is found on the apical membrane of the osteoclast, and has been reported to play a significant role in the bone resorption process. This proton pump represents an attractive target for the design of inhibitors of bone resorption which are potentially useful for the treatment and prevention of osteoporosis and related metabolic diseases. See C. Farina et al., “Selective inhibitors of the osteoclast vacuolar proton ATPase as novel bone antiresorptive agents,” DDT, 4: 163-172 (1999)), which is hereby incorporated by reference in its entirety.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89, Feb. 5, 1996 and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

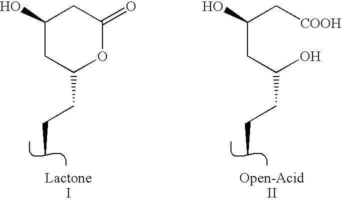

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl) aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

As used above, “integrin receptor antagonists” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁ and α₆β₄ integrins. H. N. Lode et al., PNAS USA 96: 1591-1596, 1999 have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth. αᵥβ₃ integrin receptor antagonists inhibit bone resorption through a new mechanism distinct from that of all currently available drugs. Integrins are heterodimeric transmembrane adhesion receptors that mediate cell-cell and cell-matrix interactions. The α and β integrin subunits interact non-covalently and bind extracellular matrix ligands in a divalent cation-dependent manner. The most abundant integrin on osteoclasts is αᵥβ₃ (>10⁷/osteoclast), which appears to play a rate-limiting role in cytoskeletal organization important for cell migration and polarization. The αᵥβ₃ antagonizing effect is selected from inhibition of bone resorption, inhibition of restenosis, inhibition of macular degeneration, inhibition of arthritis, and inhibition of cancer and metastatic growth.

“An osteoblast anabolic agent” refers to agents that build bone, such as PTH. The intermittent administration of parathyroid hormone (PT) or its amino-terminal fragments and analogues have been shown to prevent, arrest, partially reverse bone loss and stimulate bone formation in animals and humans. For a discussion refer to D. W. Dempster et al., “Anabolic actions of parathyroid hormone on bone,” Endocr Rev 14: 690-709, 1993. Studies have demonstrated the clinical benefits of parathyroid hormone in stimulating bone formation and thereby increasing bone mass and strength. Results were reported by R M Neer et al., New Eng J Med 344 1434-1441, 2001.

In addition, parathyroid hormone-related protein fragments or analogues, such as PTHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)] and may also have potential as anabolic agents for treating osteoporosis.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

The term “bone resorption,” as used herein, refers to the process by which osteoclasts degrade bone.

The present invention also encompasses a pharmaceutical composition useful in the treatment of osteoporosis or other bone disorders, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for a cathepsin dependent condition. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittant throughout the dosage regimen.

The compounds of the present invention can be used in combination with other agents useful for treating cathepsin-mediated conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating cathepsin-mediated conditions includes in principle any combination with any pharmaceutical composition useful for treating disorders related to estrogen functioning.

The scope of the invention therefore encompasses the use of the instantly claimed compounds in combination with a second agent selected from: an organic bisphosphonate; an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; and the pharmaceutically acceptable salts and mixtures thereof.

These and other aspects of the invention will be apparent from the teachings contained herein.

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

### Schemes

Compounds of the present invention can be prepared according to Scheme 1, as indicated below. Thus an a-amino ester may be added to a haloalkyl ketone to form an aminal which may be dehydrated to an imine in the presence of a dehydrating agent such as TiCl₄, MgSO₄ or isopropyl trifluoroacetate. Reduction of the imine with a reducing agent such as sodium cyanoborohydride or sodium borohydride provides the amine. Ester hydrolysis and amide formation with an appropriately substituted aminoacetonitrile provides compounds of the current invention. If the substituent on D system is a halogen, a palladium-catalyzed Suzuki coupling with an appropriate boronic acid provides additional compounds of the current invention. Alternatively, a copper-catalyzed or palladium-catalyzed Buchwald coupling with a suitable amine provides additional compounds of the current invention. Alternatively, a palladium-catalyzed caboxylation followed by amide formation with a suitable amine provides additional compounds of the current invention.

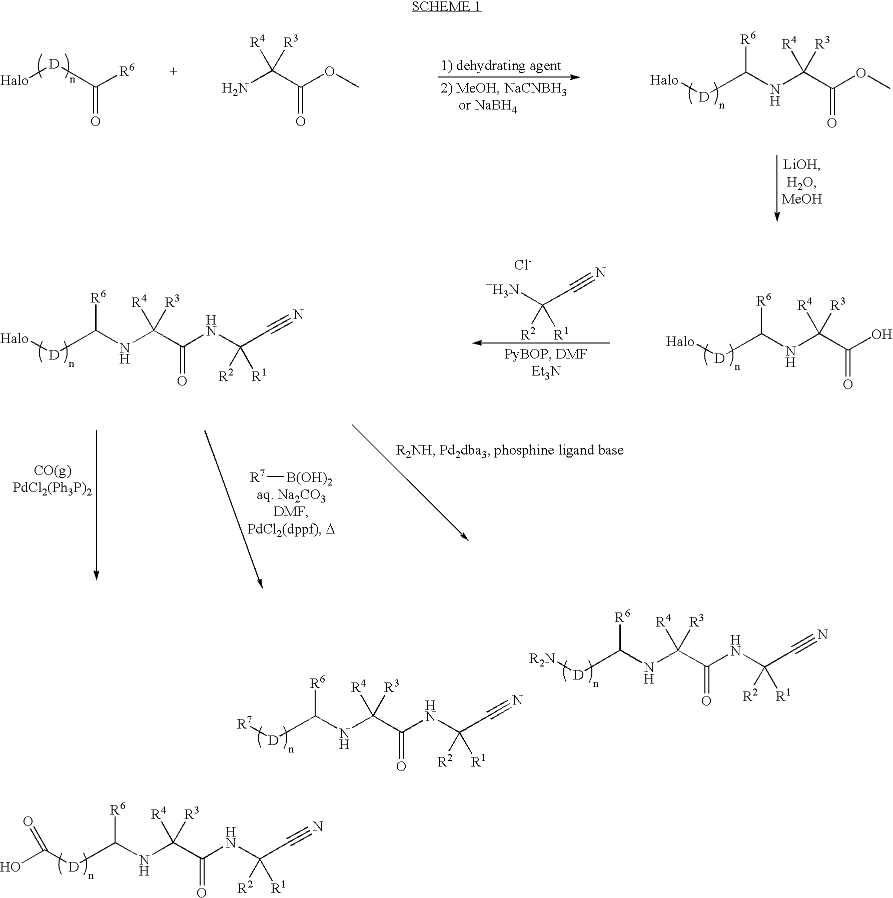

Compounds of the present invention may also be prepared according to Scheme 2, as indicated below. A ketone or aldehyde may be condensed with an amino alcohol to give a cyclic aminal. Treatment with 3 equivalents of a Grignard reagent or organolithium reagent will provide the appropriate alkylated amino alcohol. Oxidation of the alcohol with a chromium system such as a Jones oxidation or H₅IO₆/CrO₃, or alternatively by a two-step oxidation (eg oxalyl chloride/DMSO/Et₃N followed by NaClO) will provide the corresponding carboxylic acid. Peptide coupling and Suzuki reaction as described in Scheme 1 will provide compounds of the current invention.

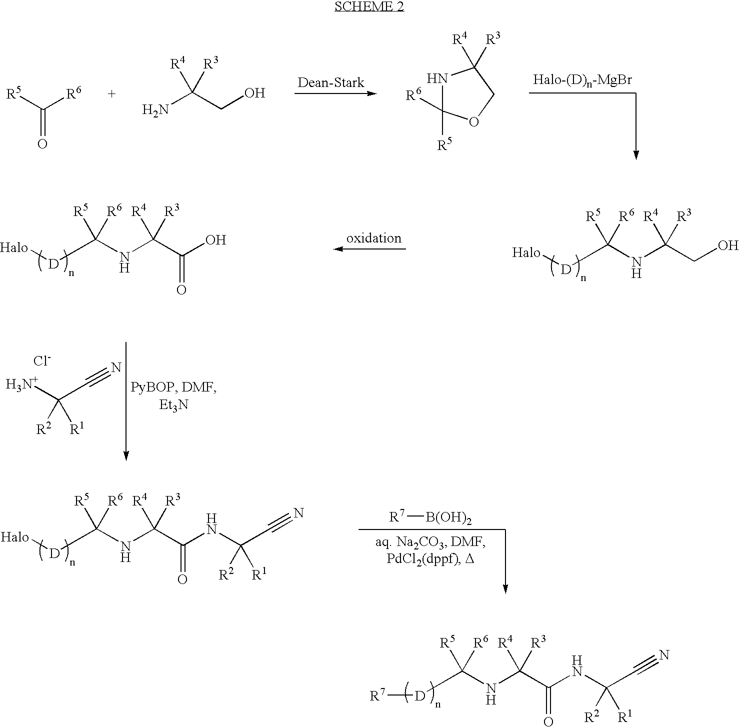

Compounds of the present invention may also be prepared according to Scheme 3, as indicated below. A ketone or aldehyde may be condensed with an amino alcohol to give an acyclic aminal. Treatment with multiple equivalents of a Grignard reagent or organolithium reagent will provide the appropriate alkylated amino alcohol. This alcohol can be converted into compounds of the current invention by the method described in Scheme 2.

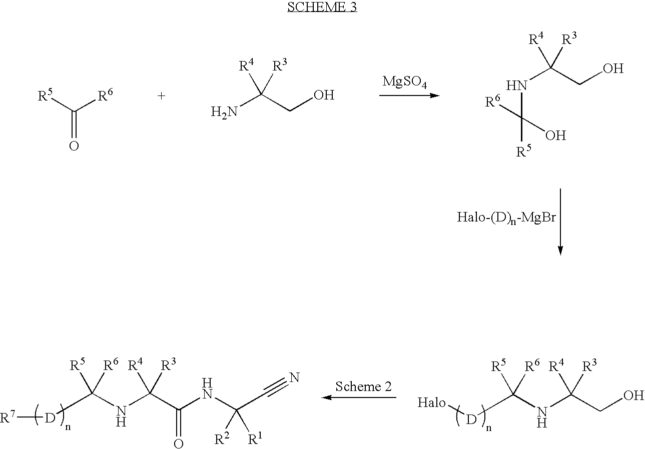

Compounds of the current invention may also be prepared according to Scheme 4. An appropriately substituted acetate may be enolized with a suitable base (including, but not limited to LDA, KHMDS, NaH or nBuLi) and treated with paraformaldehyde to generate the diol. This diol may be converted to the difluoride using a fluorinating reagent such as DAST. Hydrolysis of the ester followed by Curtius rearrangement will then provide the amine. This amine can displace an appropriately substituted alpha-bromo ester to provide the alpha-amino ester. This may be converted into compounds of the current invention by the method described in Scheme 1.

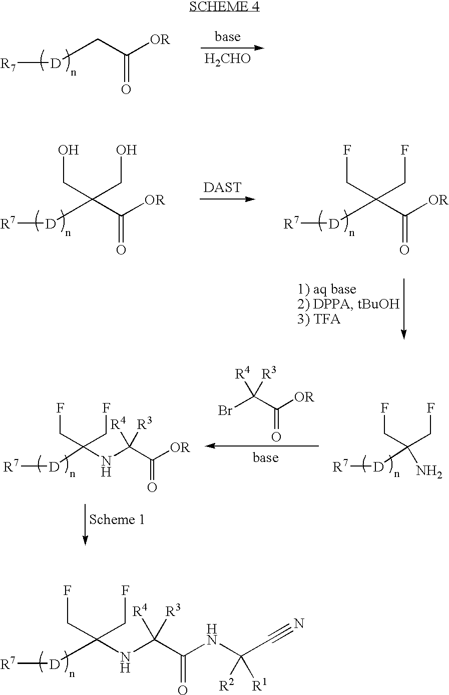

Compounds of the current invention may also be prepared according to Scheme 5, as indicated below. An aldehyde or a herniacetal may be condensed with an amino alcohol with azeotropic removal of water in which the alcohol moiety is protected with a suitable protecting group. Treatment of the resulting imine with a Grignard reagent or organolithium reagent will provide the appropriate alkylated amino alcohol. The alcohol protecting group can then be removed and the alcohol can be converted into compounds of the current invention either by the method described in Scheme 2 or by first conducting the Suzuki reaction, followed by oxidizing the alcohol with H₅IO₆ /CrO₃ and then peptide coupling.

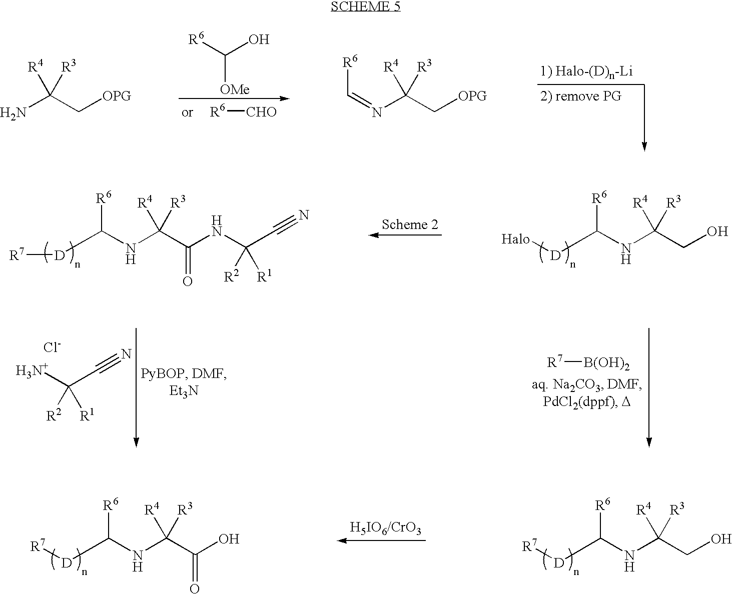

Compounds of the current invention may also be prepared according to Scheme 6, as indicated below. The peptide coupling of an alpha-amino acid described in Schemes 1, 2, or 5, with an alpha-amino amide followed by dehydration of the resulting primary amide (Voegel, J. J.; Benner, S. A. Helv. Chent Acta 1996, 79, 1863) will provide compounds of the current invention.

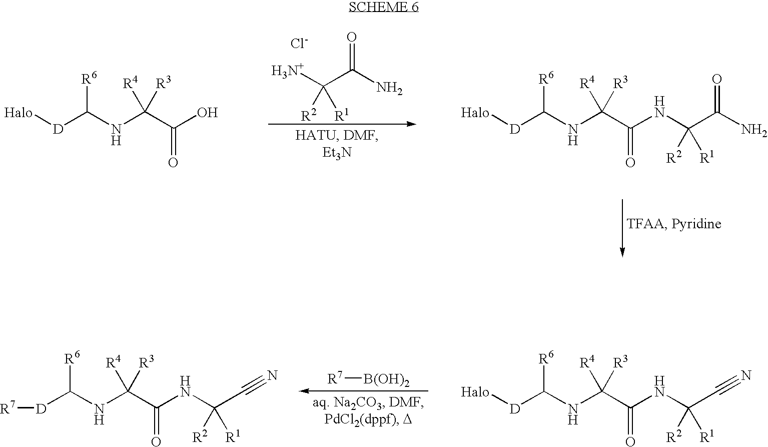

The synthesis of some of the amino alcohols used at the beginning of Schemes 2, 3 and 5 are described in Schemes 7-11. For example, the synthesis of (2S)-2-amino4-fluoro-4-methylpentan-1-ol where R=Me is described in Scheme 7 below. Starting with a suitable diprotected aspartic acid, the carboxy group can be reduced to an alcohol using standard literature procedures (i.e. mixed anyhdride formation followed by NaB reduction). A protected version of 2-amino-4-methylpentane-1,4-diol (R=Me) can then be generated by an appropriate Grignard or organolithiation reaction. Finally, the hydroxy moiety can be converted to the desired fluoro using a fluorinating agent such as DAST. The protected or unprotected version of this amino alcohol can then be converted to compounds of the current invention according to Schemes 1, 2, 3 and 5.

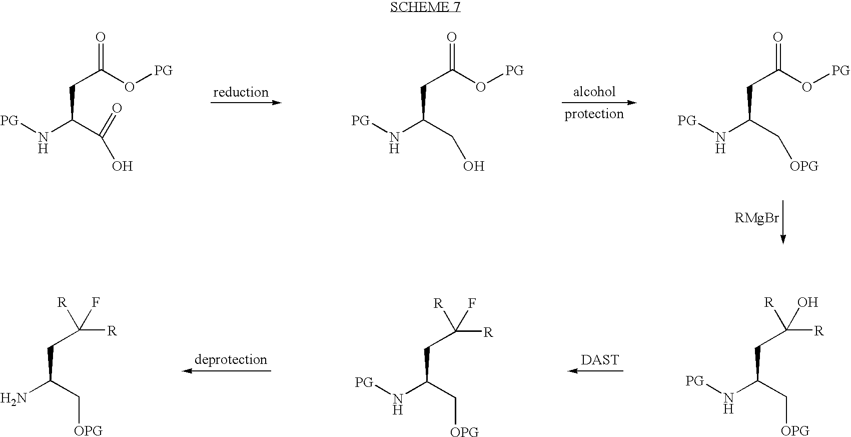

The 4-fluoroleucinol can also be synthesized according to Scheme 8. 4,5-Dehydroleucine is converted to (4S)-4-(2-methylprop-2-enyl)-1,3-oxazolidin-2-one as described in the scheme below. This intermediate is then treated with a hydrofluorination reagent such as HP-pyridine to give (4S)4-(2-fluoro-2-methylpropyl)-1,3-oxazolidin-2-one. Basic hydrolysis (i.e. Ba(OH)₂ or NaOH) then affords (2S)-2-amino4-fluoro4-methylpentan-1-ol.

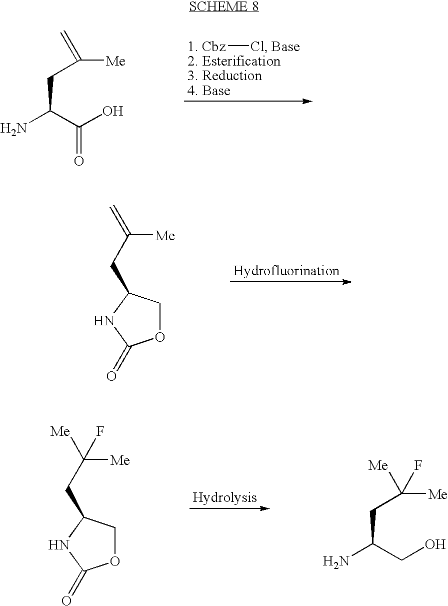

The synthesis of 4,4-difluoro-L-norvaline where R=Me is described in Scheme 9 below. Starting with a suitable diprotected serine, iodination can be carried out using a reagent such as (PhO)₃P⁺MeI⁻. Zincation of the resultant iodide may proceed using Zn.Cu couple and TMSCl. The resultant zincate can then undergo a palladium catalyzed coupling reaction with alkanoyl chloride to generate the ketone. Finally, the ketone moiety can be converted to the desired difluoro derivative using a fluorinating agent such as DAST. The protected or unprotected version of this amino acid or amino alcohol can then be converted to compounds of the current invention according to Schemes 1, 2, 3 and 5.

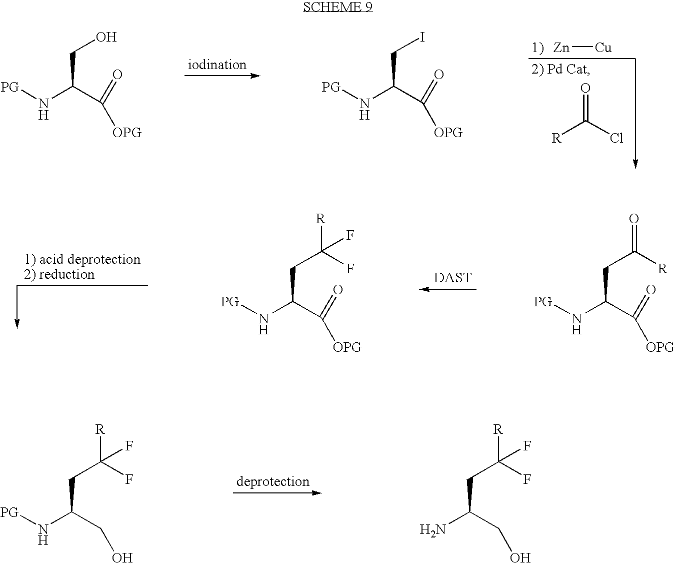

The amino alcohols used for the present invention can also be synthesized according to Scheme 10. A protected amino acid is reduced with a reducing agent such as NaBH₄ with or without an additive such as LiCl, in a solvent such as EtOH or a mixed solvent system such as EtOH and THF. The amino protecting group is then removed with the appropriate method according to the nature of the protecting group.

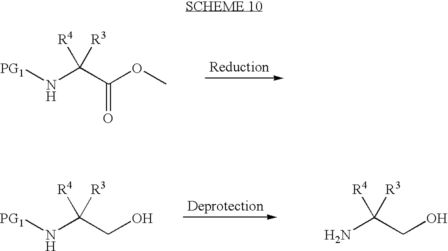

Synthesis of (2S,4S)-2-amino-5,5,5-trifluoro4-methylpentan-1-ol used in the present invention is described in Scheme 11. The N-benzoyl-5,5,5-trifluoroleucine (Ojiima, et. al. J. Org. Chem., 1989, 54, 4511-4522) can be hydrolysed with an aqueous acid such as 6M HCl under refluxing conditions. The amino acid HCl salt intermediate is then converted to the N-acetyl-5,5,5-trifluoroleucine and the amino group chiral centre is resolved by an enzymatic method (Synthetic Communications, 1996, 26, 1109-1115.). The isolated 5,5,5-trifluoro-L-leucine is then protected with a protecting group such as benzyl carbamate and the carboxylic acid group is esterified. The two diastereomers at the 4-position are then separated by flash column chromatography. One of the enantiomers, the (2S,4S) protected amino acid is then converted to the amino alcohol as described in scheme 10.

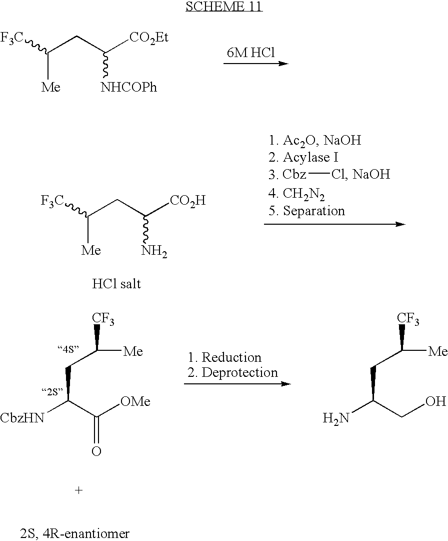

Compounds of the current invention where R⁵ is hydrogen and R⁶ is aryl or heteroaryl may also be prepared according to Scheme 12 as shown below. Condensation of an aryl or heteroaryl aldehyde with an amino alcohol in which the alcohol moiety is protected with a suitable protecting group, followed by treatment of the resulting imine with a Grignard or organolithium reagent of formula halo-(D)ₐ-Li or halo-(D)ₙ-MgX (where D is as defined in the Summary of the Invention), followed by removal of the oxygen protecting group provides the alkylated aminoalcohol. The alkylated aminoalcohol is then converted into compounds of the current invention either by the method described in Scheme 2 or by first conducting the Suzuki reaction with the boronic ester of the formula R⁷—B(OH)₂, then oxidizing the alcohol with a suitable oxidizing agent such as H₅IO₆/CrO₃ to give the acid and finally treating the acid with an aminoacetonitrile under peptide coupling conditions as described previously.

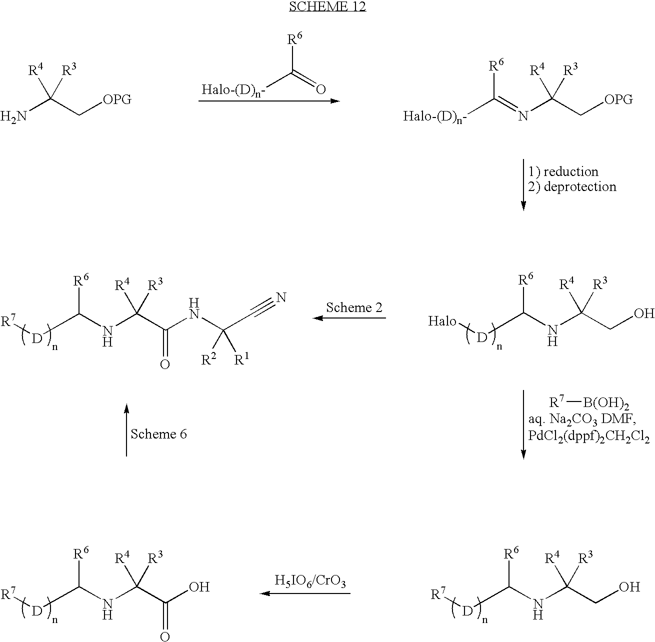

Compounds of the current invention may also be prepared according to Scheme 13, as shown below. Reaction of a suitably N-protected amino acid derivative with oxetane tosylate in the presence of sodium iodide in a suitable organic solvent such as dimethylformamide provides the corresponding oxetane ester which upon treatment with diborane provides the ortho ester. Removal of the amino protecting group affords an amine which upon condensation with an aldehyde of formula R⁶CHO (where R⁶ is aryl or heteroaryl) or a hemiacetal of formula R⁶C(OH)(OR) (where R is an alkyl group) under the reaction conditions described above provides an imine. Treatment of the imine with a Grignard or organolithium reagent under the reaction conditions described above provides an N-alkylated derivative. Removal of the ortho ester provides the corresponding carboxylic acid which is then converted into compounds of the current invention by condensation with an aminoacetonitrile under peptide coupling conditions, followed by Suzuki reaction as described above.

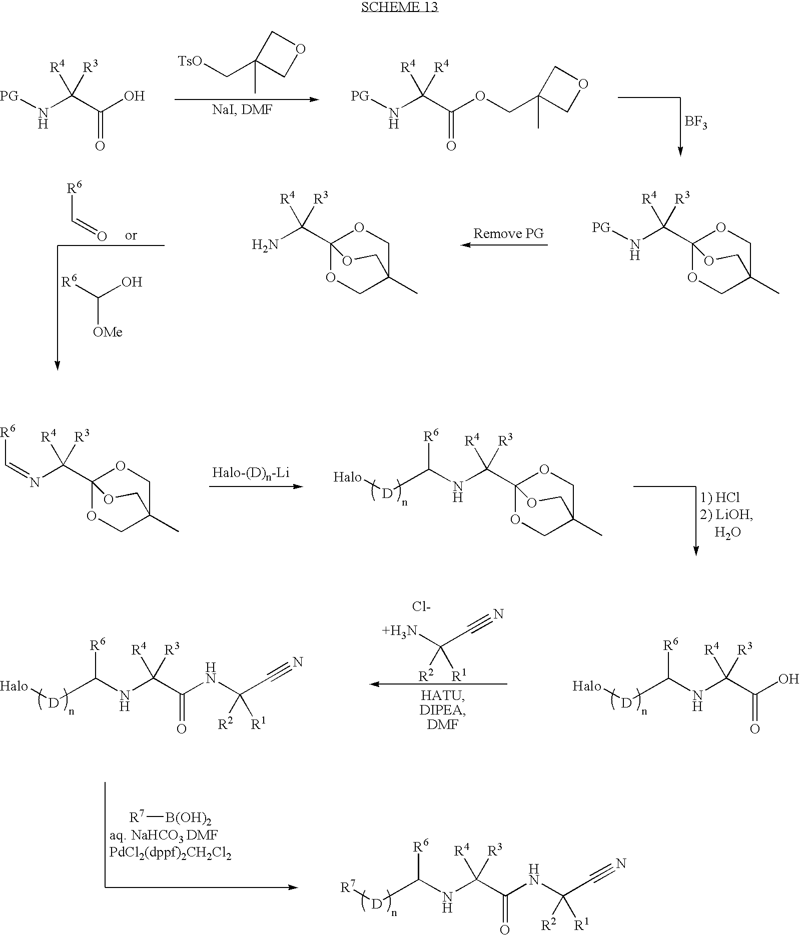

Compounds of the current invention may also be prepared as shown in Scheme 14. A aryl halide containing appropriate R¹, R², R³, R⁴ and R⁶ groups may be coupled with bis(pinacolato)diboron to give the aryl pinacolate. This may be coupled with R⁷-bromides under Suzuki conditions to provide compounds of the current invention.

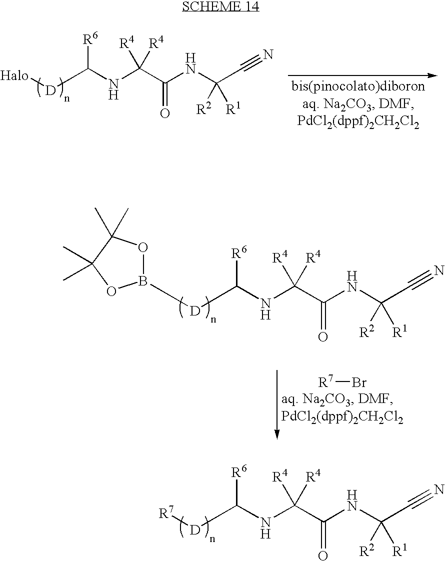

Compounds of the current invention may also be prepared according to Scheme 15, as indicated below. The peptide coupling of an appropriately substituted amino acid described in Schemes 1, 2, or 5, with an alpha-amino ester, alcohol or ketone will provide compounds of the current invention. In the case of an alpha-amino alcohol, oxidation of the product alcohol will provide a ketone which is a compound of the current invention. In the case of an alpha-amino ester, hydrolysis of the product ester followed by amide formation with a suitable amine will provide amides which are compounds of the current invention.

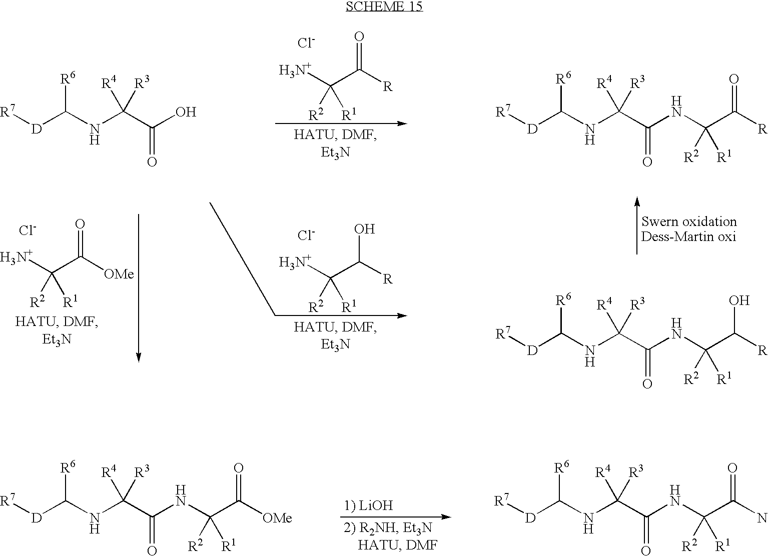

Acids shown in Schemes 1, 2, 6 and 15 may also be prepared as shown in Scheme 16. An appropriately substituted benzyl bromide, iodide or triflate (which may be chiral or racemic) may be coupled with an alpha amino ester under basic conditions. Hydrolysis with aqueous base then provides the acid which can be converted into examples of the current invention.

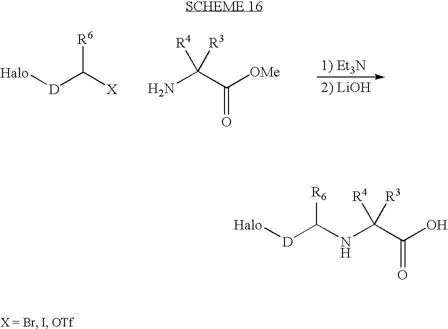

The following examples describe the synthesis of selected compounds of the present invention. These examples are illustrative of the invention claimed herein, and are not to be interpretated as limiting the scope of the invention. Table 1 describes how specific examples, when bound in the active site of a cathepsin for which it is active (e.g. Cathepsin K), meet the distance criteria as described herein.

### EXAMPLE 1

### Synthesis of N¹-(cyanomethyl)-N²-(2,2.2-trifluoro-1-phenolethyl)-L-leucinamide

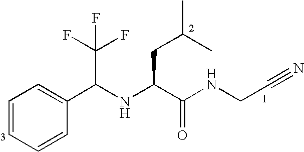

To a solution of L-leucine methyl ester hydrochloride (975 mg, 5.37 mmol) in dichloromethane (30 mL) was added 2,2,2-trifluoroacetophenone (0.75 mL, 5.34 mmol) and diisopropylethylamine (3.5 mL, 20 mmol). TiCl₄ (0.55 mL, 5.0 mmol) in 0.45 mL dichloromethane was added dropwise, and the mixture was stirred overnight. Additional TiCl₄ (0.4 mL, 3.6 mmol) was then added and the mixture was stirred 3h. A solution of NaCNBH₃ (1050 mg, 16.7 mmol) in MeOH (20 mL) was added and the mixture was stirred 2 h. Poured into 1N NaOH and extracted with ethyl acetate (2×). The organic phase was washed with 1N NaOH and brine, then dried over MgSO₄ and evaporated. Purification by ISCO column chromatography (gradient 30% to 90% ethyl acetate/hexanes) provided methyl N-(2,2,2-trifluoro-1-phenylethyl)-L-leucinate.

To a room temperature solution of methyl N-(2,2,2-trifluoro-1-phenylethyl)-L-leucinate (150 mg, 0.50 mmol) in 2:1 THF/MeOH was added 1M LiOH. The mixture was stirred overnight and concentrated. The residue was partitioned between ethyl acetate and pH 3.5 phosphate buffer. The organic phase was washed with brine, dried over MgSO₄ and concentrated to give N-(2,2,2-trifluoro-1-phenylethyl)-L-leucine.

A mixture of N-(2,2,2-trifluoro-1-phenylethyl)-L-leucine (149 mg, 0.50 mmol), aminoacetonitrile hydrochloride (102 mg, 1.1 mmol) and PyBOP (260 mg, 0.50 mmol) was dissolved in DMF (5 mL). Triethylamine (0.3 mL, 2.1 mmol) was added and the mixture was stirred overnight, then poured into pH 3 phosphate buffer and extracted with 3:1 ether/ethyl acetate. The organic phase was washed with saturated aqueous NaHCO₃ and brine, dried over MgSO₄ and evaporated. Purification by ISCO column chromatography (gradient 20% to 50% ethyl acetate/hexanes) provided N¹-(cyanomethyl)-N²-(2,2,2-trifluoro-1-phenylethyl)-L-leucinamide as a 1:1 mixture of diastereomers. MS (+APCI): 313.9 [M+1].

### EXAMPLE 2

### Synthesis of N²-[1-(4-bromophenyl)-2,2,2-trifluoroethyl]-N¹-(cyanomethyl)-L-leucinamide

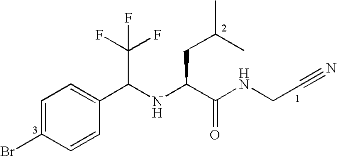

Using the method of Example 1, N²-[1-(4-bromophenyl)-2,2,2-trifluoroethyl]-N¹-(cyanomethyl)-L-leucinamide was prepared.

MS (−ESI): 403.9, 405.9 [M−1]⁻

### EXAMPLE 3

### Synthesis of N¹-(Cyanomethyl)-N²-{[4′-(methylsulfonyl)-1,1′-biphenyl-4-yl][4-(methylsulfonyl)phenyl]methyl}-L-Leucinamide

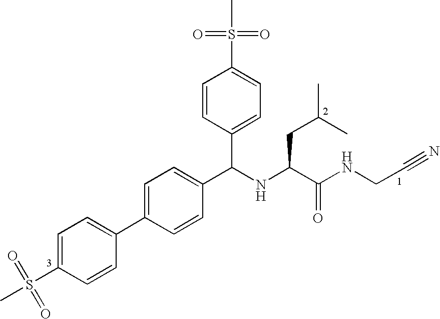

### Step 1: Methyl N-{(4-bromophenyl)[4-(methylsulfonyl)phenyl]methylene}-L-leucinate

A solution of (4-bromophenyl)[4-(methylsulfonyl)phenyl]methanone (202 mg, 0.59 mmol), L-leucine methyl ester hydrochloride (328 mg, 2.0 mmol) and camphor sulfonic acid (52 mg, 0.22 mmol) in toluene was refluxed for 18 hours using a Dean-Stark trap. The solvent was removed in vacuo and the resulting residue was purified by chromatography using EtOAc and hexane as eluant to give a 1:1 mixture of the title compound and the starting (4-bromophenyl)[4-(methylsulfonyl)phenyl]methanone.

### Step 2: Methyl N-{(4-bromophenyl)[4-(methylsulfonylphenyl]methyl}-L-leucinate

To a solution of a 1:1 mixture of methyl N-{(4-bromophenyl)[4-(methylsulfonyl)phenyl]methylene}leucinate and (4-bromophenyl)[4-(methylsulfonyl)phenyl]methanone from step 1 (185 mg, ˜0.2 mmol) in acetic acid/methanol (1:3, 4 mL) was added sodium borohydride (˜400 mg) by portions every 30 min over 2 days (addition was stopped during the night) using a solid addition funnel. The reaction mixture was partitioned between EtOAc and water, the organic layer was dried over Na₂SO₄ and concentrated. The resulting mixture was purified by chromatography using EtOAc and hexane as eluant. Methyl N-{(4-bromophenyl)[4-(methylsulfonyl)phenyl]methyl}-L-leucinate was obtained as a colorless gum and (4-bromophenyl)[4-(methylsulfonyl)phenyl]methanol was obtained as a white solid.

### Step 3: N-{(4-bromophenyl)[4-(methylsulfonyl)phenl]methyl}-L-leucine

To a solution of methyl N-{(4-bromophenyl)[4-(methylsulfonyl)phenyl]methyl}-L-leucinate from step 2 (81 mg, 0.17 mmol) in THF (1 mL) and MeOH (0.5 mL) was added 1N LiOH (0.3 mL, 0.3 mmol). The resulting mixture was stirred at room temperature for 18 hours and then partitioned between EtOAc and water+1N HCl (0.5 mL). The organic layer was dried over Na₂SO₄, filtered and concentrated in vacuo to give the title compound as a colorless gum.

### Step 4: N²-{(4-bromophenyl)[4-(methylsulfonyl)phenyl]methyl}-N¹-(cyanomethyl)-L-leucinamide

To a solution of N-{(4-bromophenyl)[4-(methylsulfonyl)phenyl]methyl}-L-leucine from step 3 (76 mg, 0.17 mmol), HATU (146 mg, 0.38 mmol), aminoacetonitrile hydrochloride (52 mg, 0.56 mmol) in DMF (1.1 mL) cooled to −10° C., was added N,N-diisopropylethylamine (0.13 mL, 0.75 mmol). The reaction was allowed to proceed at room temperature for 18 h and it was partitioned between EtOAc and water. The organic layer was dried over Na₂SO₄, filtered and concentrated in vacuo. The crude product was purified by chromatography using EtOAc and hexane as eluant to give the title compound as a colorless gum.

### Step 5: N¹-(cyanomethyl)-N²-{[4-(methylsulfonyl)phenyl][4′-(methylthio)-1,1′-biphenyl-4-yl]methyl}-L-leucinamide

A heterogeneous mixture of N²-{(4-bromophenyl)[4-(methylsulfonyl)phenyl]methyl)-N¹-(cyanomethyl)-L-leucinamide from step 4 ( 72 mg, 0.15 mmol), 4-(methylthio)phenylboronic acid (37 mg, 0.22 mmol) in ethylene glycol dimethyl ether (1 mL) and 2M aqueous sodium carbonate was degassed under vacuum and purged with nitrogen.

To this mixture was added [1,1′-bis(diphenylphosphino)ferrocene]dichloropalladium(II), dichloromethane complex (19 mg, 0.023 mmol), followed by degassing and purging with nitrogen. The reaction mixture was heated at 85° C. for 16 hours with efficient stirring. The reaction mixture was partitioned between EtOAc and aqueous NH₄OAc 25% w/v. The organic layer was dried over Na₂SO₄, filtered and concentrated in vacuo. The crude product was purified by chromatography using EtOAc and hexane as eluant to give the title compound as a colorless gum.

### Step 6: N¹-(Cyanomethyl)-N²-{[4′-(methylsulfonyl)-1,1′-biphenyl-1-yl][4-(methylsulfonyl)phenyl]methyl}-L-Leucinamide

To a solution of N¹-(cyanomethyl)-N²-{[4-(methylsulfonyl)phenyl][4′-(methylthio)-1,1′-biphenyl4-yl]methyl}-L-leucinamide (63 mg, 0.12 mmol), sodium tungstate dihydrate (2 mg, 0.006 mmol), tetrabutylammonium hydrogensulfate (4 mg, 0.01 mmol) was added a solution of 30% w/v aqueous hydrogen peroxide (100 μL, 0.9 mmol) and the resulting mixture was stirred at room temperature for 10 min. The reaction mixture was partitioned between EtOAc and water+1M NaHSO₃ (˜3:1). The organic layer was dried over Na₂SO₄, filtered and concentrated in vacuo. The crude product was purified by chromatography using EtOAc and hexane as eluant to give the title compound as a colorless gum.

MS (+ESI): 568.2 [M+1]⁺.

### EXAMPLE 4

### Synthesis of N¹(cyanomethyl)-N²{(1S)-2,2.2-trifluoro-1-[4′-(methylsulfonyl)-1,1′-biphenyl-4yl]ethyl}-L-leucinamide

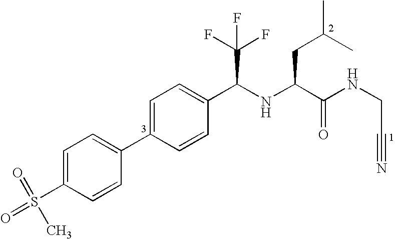

### Step 1: (2S)-1-{[tert-butyl(dimethy)silyl]oxy}-4-methylpentan-2-amine

To a room temperature dichloromethane (100 mL) solution of L-leucinol (6.0 g) was added triethylamine (11 mL), DMAP (0.1 g) and t-butyldimethylsilyl chloride (8.5 g). The mixture was stirred at room temperature for 2 hours and then water was added. The organic layer was separated and the aqueous further extracted with dichloromethane. The combined organic layers were washed with brine, dried with magnesium sulfate and the solvent was removed in vacuo to yield the title compound, a residue which was used as such in the next reaction. ¹H NMR (CD₃COCD₃) δ 3.48(m, 2H), 3.32(m, 1H), 2.76(m, 1H), 1.78(m, 1H), 1.22-1.02(m, 2H), 0.88(m, 15H), 0.06(s, 6H).

### Step 2: (2S)-1-{[tert-butyl(dimethyl)silyl]oxy}-4-methyl-N-[(1E)-2,2,2-trifluoroethylidene]pentan-2-amine

A toluene (300 mL) solution of (2S)-1-{[tert-butyl(dimethyl)silyl]oxy}-4-methylpentan-2-amine from Step 1 (50 g) and tifluoroacetaldehyde methyl hemiacetal ( 35 mL) was heated to reflux for 16 hours during which time water was collected in a Dean-Stark trap. The solvent was evaporated in vacuum and the residue was purified on SiO₂ using hexanes and ethyl acetate (9:1) as eluant to yield the title compound.

¹H NMR (CD₃COCD₃) δ 7.88(m, 1H), 3.76-3.45(m, 3H), 1.60-1.25(m, 3H), 0.88(m, 15H), 0.006(s, 3H), 0.04(s, 3H).

### Step 3: (2S)-2-{[(1S)-1-(4-bromophenyl)-2,2,2-trifluoroethyl]amino}-4-methylpentan-1-ol

n-BuLi (2.5 M in hexanes, 42 mL) was added to a −70° C. THF (400 mL) solution of 1,4-dibromobenzene (25.8 g) and the mixture was stirred for 25 minutes. A TBI (30 mL) solution of (2S)-1-{[tert-butyl(dimethyl)silyl]oxy}-4-methyl-N-[(1E)-2,2,2-trifluoroethylidene]pentan-2-amine (31 g) was then added dropwise and the mixture was stirred for 1.5 hour. It was then poured slowly into a mixture of ethyl acetate (500 mL), water (2 L), ice (300 g) and ammonium chloride (100 g) under vigorous stirring. The organic layer was separated and the aqueous further extracted with ethyl acetate (2×500 mL). The combined organic layers were washed with brine, dried with magnesium sulfate and the solvent was removed in vacuo to yield a residue, which was used as such. The residue from above was dissolved in THP (250 mL) and the solution was cooled to 0° C. A 1 M THF solution of t-butylammonium fluoride (110 mL) was added dropwise and the mixture was reacted for 4 hours. It was poured into ethyl acetate (300 mL), water (2 L) and ammonium chloride (100 g) under vigorous stirring. The organic layer was separated and the aqueous further extracted with ethyl acetate (2×100 mL). The combined organic layers were washed with brine, dried with magnesium sulfate and the solvent was removed in vacuo to yield a residue which was purified purified on SiO₂ using a gradient of ethyl acetate and hexanes (1:5 to 1:4) as eluant to yield the title compound.

¹H NMR (CD₃COCD₃) δ 7.6(2H, d), 7.45(2H, d), 4.55(1H, m), 3.65-3.7(1H, m), 3.5-3.55(1H, m), 3.25-3.35(1H, m), 2.6-2.7(1H, m), 2.25-2.35(1H, m), 1.65-1.75(1H, m), 1.3-1.4(1H, m), 1.2-1.3(1H, m), 0.75-0.9(6H, dd).

### Step 4: (2S)-4-methyl-2-({(1S)-2,2,2-trifluoro-1-[4′-(methylthio)-1,1′-biphenyl-4-yl]ethyl}amino)pentan-1-ol

A stream of nitrogen was passed through a suspension made of the bromide from Step 3 (27.7 g), 4-(methylthio)phenylboronic acid (15.7 g), 2 M Na₂CO₃ (100 mL) and n-propanol (500 mL) for 15 minutes. A 1:3 mixture (3.5 g) of Pd(OAc)₂ and PPh₃ was then added and the reaction was warmed to 70° C. and stirred under nitrogen for 8 hours. The mixture was cooled to room temperature, diluted with ethylacetate (500 mL) and poured over water (2 L) and ice (500 g). The ethyl acetate layer was separated and the aqueous further extracted with ethyl acetate (200 mL). The combined ethyl acetate extracts were washed with 0.5 N NaOH (2×200 mL), with aqueous NH₄Cl, brine and dried with magnesium sulfate. Removal of the solvent left a residue that was purified by chromatography on SiO₂ using a gradient of ethyl acetate and hexanes (1:4 to 1:3) and again with acetone and toluene (1:10). The residue was dissolve in hot hexanes (200 mL) and the solution was allowed to cool to 0° C. under stirring. The obtained solid was filtered and dried to yield the title compound.

¹H NMR (CD₃COCD₃) δ 7.7(2H, d), 7.65(2H, d), 7.6(2H, d), 7.35(2H, d), 4.5-4.6(1H, m), 3.7(1H(OH), m), 3.5-3.6(1H, m), 3.3-3.4(1H, m), 2.7(1H, m), 2.5(3H, s), 2.3-2.4(1H(NH), m), 1.65-1.75(1H, m), 1.2-1.4(3H, m), 0.8-0.9(6H, dd).

### Step 5: (2S)4-methyl-2-({(S)-2,2,2-trifluoro-1-[4′-(methylsulfonyl)-1,1═-biphenyl-4-yl]ethyl}amino)pentan-1-ol

To a 0° C. solution of the sulfide (19 g) from Step 4 in toluene (400 mL) was added Na₂WO₄.2H₂O (0.16 g) and Bu₄NHSO₄ (0.81 g). Then 30% hydrogen peroxide (12.2 mL) was slowly added and the mixture was stirred at room temperature for 4.5 hours. The mixture was poured slowly on a mixture of ice, dilute aqueous sodium thiosulfate and ethyl acetate. The organic layer was separated and the aqueous further extracted with ethyl acetate (2×100 mL). The combined organic layers were washed with brine, dried with magnesium sulfate and the solvent were removed in vacuo to yield a residue which was purified purified on SiO₂ using ethyl acetate and hexanes (1:1) as eluant to yield the product.

¹H NMR (CD₃COCD₃) δ 8.05(2H, d), 8.0(2H, d), 7.85(2H, d), 7.7(2H, d), 4.6-4.7(1H, m), 3.75(1H, m), 3.6(1H, m), 3.35-3.45(1H, m), 3.2(3H, s), 2.7-2.8(1H, m), 2.35-2.45(1H, m), 1.7-1.8(1H, m), 1.2-1.5(2H, m), 0.8-0.95(6H, dd).

### Step 6: Preparation of N-{(1S)-2,2,2-trifluoro-1-[4′-(methylsulfonyl)-1,1′-biphenyl-4-yl]ethyl}-L-leucine

A suspension of H₅IO₆/CrO₃ (529 mL of 0.44 M in CH3CN; see Note below) was cooled to 0° C. and a solution of the alcohol from Step 5 (20 g) in CH₃CN (230 mL) was added dropwise. The mixture was stirred at 0-5° C. for 3.5 hours. It was poured into pH 4 Na₂HPO₄ (1.5 L) under vigorous stirring and the mixture was extracted with diethyl ether (3×250 mL). The combined ether extracts were washed with water and brine (1:1), with dilute aqueous NaHSO₃ and brine. The organic extract was dried with sodium sulfate, filtered and the solvents were evaporated to dryness to yield a residue that was split into two batches for the following purification.

The crude acid from above (10 g) was dissolved in isopropyl acetate (250 mL) and extracted into cold 0.1 N NaOH (3×250 mL). The combined extracts were washed with diethyl ether (250 mL) and then slowly acidified with 6 N HCl to pH 4. The carboxylic acid was extracted with isopropyl acetate (2×250 mL) and the isopropyl acetate layer dried and concentrated to yield the product essentially pure and used as such in the next step.

Note: The oxidizing reagent (H₅IO₆/CrO₃) was prepared as described in Tetrahedron Letters 39 (1998) 5323-5326 but using HPLC grade CH₃CN (contains 0.5% water); no water was added.

¹H NMR (CD₃COCD₃) δ 8.05(2H, d), 7.95(2H, d), 7.8(2H, d), 7.65(2H, d), 4.45-4.55(1H, m), 3.55-3.6(1H, m), 3.2(3H, s), 2.8-3.0(broad m, NH/(OH)1.95-2.05(1H, m), 1.55-1.6(2H, m), 0.9-1.0(6H, m).

### Step 7: Preparation of N¹(cyanomethyl)-N²{(1S)-2,2.2-trifluoro-1-[4′-(methylsulfonyl-1,1′-biphenyl-4-yl]ethyl}-L-leucinamide

To a DMF (200 mL) solution of the acid from Step 7 (9 g) was added benzotriazol-1-yloxytris(dimethylamino)phosphonium hexafluorophosphate (11.6 g), aminoacetonitrile hydrochloride (3.94 g) and the mixture was cooled to 0° C. Triethylamine (9.9 mL) was added dropwise and the mixture warmed to room temperature and stirred for 16 hours. It was poured into ice and saturated aqueous sodium bicarbonate and extracted with diethyl ether (3×100 mL). The combined extracts were washed with brine, dried with magnesium sulfate and the solvent removed in vacuo. The residue was purified by chromatography on SiO₂ using ethyl acetate and hexanes (1:1). The title compound was then stirred in diethyl ether for 16 hours, filtered and dried (mp 140.5° C.).

¹H NMR (CD₃COCD₃) δ 8.0(2H, d), 7.95(2H, d), 7.8(2H, d), 7.65(2H, d), 4.35-4.45(1H, m), 4.1-4.2(2H, m), 3.45-3.55(1H, m), 3.15(3H, s), 2.65-2.7(1H, m), 1.85-1.95(1H, m), 1.4-1.6(2H, m), 0.85-0.95(6H, m).

### EXAMPLE 5

### Preparation of N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide

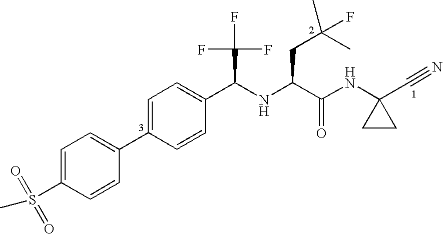

### Step 1: Benzyl(3S)-3-[(tert-butoxycarbonyl)amino]-4-hydroxybutanoate

N-(tert-Butoxycarbonyl)-L-aspartic acid 4-benzyl ester (30 g) was dissolved in dimethoxyethane (90 mL) and the solution was cooled to −5° C. N-Methylmorpholine (10.32 mL) was added followed by isobutyl chloroformate (12.7 mL) in such a way to keep the temperature below −10° C. The mixture was aged for 0.5 hour. The solids were quickly filtered and washed with dimethoxyethane (90 mL). To the filtrate cooled to −50° C. was carefully added sodium borohydride (4.4 g) as a solution in water (45 mL) in such a way to keep the temperature between −30° C. and −15° C. After all the hydride had been added, water (500 mL) was added in such a way to maintain the temperature below −15° C. The suspension was filtered, the solid washed with water (400 mL) and dried to yield benzyl (3S)-3-[(tert-butoxycarbonyl)amino]-4-hydroxybutanoate.

¹H NMR (CD₃COCD₃) δ 7.3-7.45(5H, m), 5.85-5.95(1H, NH), 5.15(2H, s), 3.95-4.1(2H, m), 3.5-3.7(2H, m), 2.55-2.75(2H, m), 1.4(9H, s).

### Step 2: Benzyl[(4S)-2-oxo-1,3-oxazolidin-4-yl]acetate

To the alcohol (95.7 g) from Step 1 dissolved in dichloroethane (925 mL) was added pyridine (625 mL) and the mixture was cooled to 0-5° C. Anhydrous p-toluenesulfonic anhydride (105.7 g.) was added and the mixture was warmed to room temperature and stirred for 1 hour. It was then heated to 90° C. for 2 hours. The mixture was cooled, diluted with dichloromethane (1000 mL) and washed with 1N HCl (3×600 mL). The organic layer was washed with brine, dried with sodium sulfate and the solvents were removed ill vacuo. The residue was purified by chromatography on SiO₂ using ethyl acetate and hexanes in a 1:1 ratio followed by ethyl acetate to yield benzyl [(4S)-2-oxo-1,3-oxazolidin-4-yl]acetate.

¹H NMR (CD₃SOCD₃) δ 7.8(1H, NH), 7.3-7.45(5H, m), 5.05-5.15(2H, m), 4.4-4.5(1H, m), 4.1-4.2(1H, m), 4.0-4.05(1H, m), 3.6-3.8(2H, m).

### Step 3: (4S)-4-(2-Hydroxy-2-methylpropyl)-1,3-oxazolidin-2-one

Methylmagnesium bromide (227 mL of 3M solution in diethyl ether) was added to a mixture of toluene (340 mL) and THF (340 mL) at −20° C. The ester from Step 2 (40 g) as a warm THF solution (170 mL) was then added dropwise maintaining the temperature below −10° C. and the mixture was aged for 2 hours. The mixture was then slowly added to a mixture of water (1000 mL) and acetic acid (200 mL) and the mixture was stirred for 2 hours at room temperature. The aqueous layer was separated and the organic extracted with water (2×200 mL). The product was extracted from the combined aqueous layers using dichloromethane and a continuous extractor. The dichloromethane extract was evaporated to dryness with the help of heptane. The residue was purified by chromatography on SiO₂ using ethanol and dichloromethane (1:30) to yield (4S)-4-(2-hydroxy-2-methylpropyl)-1,3-oxazolidin-2-one.

¹H NMR (CD₃COCD₃) δ 6.1-6.4(1H, NH), 4.45-4.55(1H, m), 4.1-4.2(1H, m), 3.95-4.05(1H, m), 3.7(1H, s), 1.65-1.85(2H, m), 1.25(6H, m).

### Step 4: (4S)-4-(2-Fluoro-2-methylpropyl)-1,3-oxazolidin-2-one

The alcohol (47.8 g.) from Step 3 as a dichloromethane (100 mL) solution was added to a −70° C. solution of (diethylamino)sulfur trifluoride (48.5 g.) in dichloromethane (500 mL). The mixture was warmed to room temperature and stirred for 1 hour. It was then carefully added to a 0° C. mixture of saturated aqueous NaHCO₃ (800 mL). The organic layer was separated and washed with saturated aqueous NaHCO₃. The aqueous was further extracted with dichloromethane (100 mL) and the combined dichloromethane layers were dried and concentrated. The residue was purified by chromatography on SiO₂ using ethyl acetate and hexanes (1:5) followed by ethyl acetate to yield (4S)-4-(2-fluoro-2-methylpropyl)-1,3-oxazolidin-2-one.

¹H NMR (CD3SOCD₃) δ 7.6(1H, NH), 4.44.5(1H, m), 3.954.05(1H, m), 3.9-3.95(1H, m), 1.8-1.95(2H, m), 1.25-1.4(6H, 2s).

### Step 5: (2S)-2-Amino-4-fluoro-4-methylpentan-1-ol

To the fluoro derivative (21.0 g) from Step 4 dissolved in 90% aqueous ethyl alcohol (216 mL) was added potassium hydroxide (21.9 g). The mixture was heated at reflux for 4 hours and cooled to room temperature. The mixture was then concentrated and co-evaporated with toluene (3×300 mL). The residue was dissolved in dichloromethane (500 mL) and stirred for 0.5 hour. The suspension was filtered through celite and the celite was washed with dichloromethane (3×100 mL). The filtrate was concentrated to dryness to yield (2S)-2-amino-4-fluoro-4-methylpentan-1-ol.

¹H NMR (CD₃OD) δ 3.4-3.5(1H, m), 3.2-3.3(1H, m), 3.0-3.1(1H, m), 1.5-1.7(2H, m), 1.35(3H, s), 1.3(3H, s).

### Step 6: (2S)-1-{[tert-butyl(dimethyl)silyl]oxy}-4-fluoro-4-methylpentan-2-amine

The amino alcohol (21.0 g) from Step 5 was dissolved in dichloromethane (300 mL) and the solution was cooled to 0° C. 4-(Dimethylamino)pyridine (0.051 g.) and tert-butyldimethylsilyl chloride (21 g.) were added followed by triethylamine (25 mL). The mixture was stirred at room temperature overnight. The reaction mixture was slowly poured into 0° C. saturated aqueous ammonium chloride and extracted with dichloromethane (3×300 mL). The organic layer was washed with brine, dried with sodium sulfate and the solvents were removed in vacuo to yield (2S)-1-{[tert-butyl(dimethyl)silyl]oxy}-4-fluoro-4-methylpentan-2-amine.

¹H NMR (CD₃OD) δ 3.6-3.65(1H, m), 3.4-3.5(1H, m), 3.1-3.2(1H, m), 1.6-1.8(2H, m), 1.35-1.45(6H, m), 0.93(9H, s), 0.1(6H, s).

### Step 7: (2S)-1-{[tert-butyl(dimethyl)silyl]oxy}-4-fluoro-4-methyl-N-[(1E)-2,2,2-trifluoroethylidene]pentan-2-amine

To the amine (31.5 g) from Step 6 dissolved in benzene (126 mL) was added trifluoroacetaldehyde methyl hemiacetal (21.6 mL.). The solution was heated at reflux overnight using a Dean-Stark trap to collect water. The reaction mixture was cooled to room temperature and concentrated to dryness. The residue was purified on SiO₂ using 4% of ethyl acetate in hexanes to yield (2S)-1-{[tert-butyl(dimethyl)silyl]oxy}-4-fluoro4-methylpentan-2-amine.

¹H NMR (CD₃COCD₃) δ 7.9-7.95(1H, m), 3.75-3.85(1H, m), 3.7-3.75(1H, m), 3.53-3.6(1H, m), 1.9-2.0(2H, m), 1.3-1.4(6H, m), 0.9(9H, s), 0.1(3H, s), 0.05(3H, s).

### Step 8: (2S)-2-{[(1S)-1-(4-Bromophenyl)-2,2,2-trifluoroethylamino}-4-fluoro-4-methylpentan-1-ol

To a −75° C. solution of 1,4-dibromobenzene (0.26 g) in THF (4 mL) was added n-BuLi (0.42 mL of a 2.5M hexanes solution) and the mixture was aged for 20 minutes. The imine (0.329 g.) from Step 7 in THF (2 mL) was added and the mixture was aged 2 hours. The mixture was then added to a mixture of water (50 mL), NH₄Cl (1 g.) and crushed ice. It was extracted with ethyl acetate (2×25 mL) and the combined ethyl acetate layers were dried and evaporated to dryness.

The same procedure was repeated but using dibromobenzene (1.2 g.), n-BuLi (1.84 mL) and the imine (1.38 g.) and the reaction mixture was treated as above. The combined residues from both preparations were dissolved in TBF (10 mL) and cooled to 0° C. n-Tetrabutylammonium fluroride (6 mL from a 1M THF solution) was added and the mixture was stirred at +5° C. for 16 hrs. It was poured into a mixture of water (50 mL), ammonium chloride (1 g.) and crushed ice and the organic layer was separated. The aqueous was further extracted with ethyl acetate ( 2×15 mL) and the combined organic layers were dried and concentrated. The residue was purified on SiO₂ using ethyl acetate and hexanes (1:5) to yield (2S)-2-{[(1S)-1-(4-bromophenyl)-2,2,2-trifluoroethyl]amino}-4-fluoro-4-methylpentan-1-ol.

¹H NMR (CD₃COCD₃) δ 7.65(2H, m), 7.5(2H, m), 4.5-4.6(1H, m), 3.8(1H, m), 3.6(1H, m), 3.33-3.4(1H, m), 2.85-2.0(1H, m), 2.55(1H, m), 1.7-1.9(2H, s), 1.3-1.4(6H, m).

### Step 9: N-[(1S)-1-(4-bromophenyl)-2,2,2-trifluoroethyl]-4-fluoro-L-leucine

A suspension of H₅IO₆/CrO₃ (66 mL of 0.44 M in CH₃CN; Note) was cooled to 0° C. and a solution of the alcohol from Step 8 (1.55 g) in CH₃CN (5 mL) was added dropwise. The mixture was stirred at 0-5° C. for 3.5 hours. It was poured into pH 4 Na₂HPO₄ (200 mL) under vigorous stirring and the mixture was extracted with diethyl ether (3×50 mL). The combined ether extracts were washed with water and brine (1:1) followed by dilute aqueous NaHSO₃ and brine. It was dried with sodium sulfate, filtered and the solvents were evaporated to dryness to yield of N-[(1S)-1-(4-bromophenyl)-2,2,2-trifluoroethyl]4-fluoro-L-leucine used as such in the next step.

Note. The oxidizing reagent (H₅IO₆/CrO₃) was prepared as described in Tetrahedron Letters 39 (1998) 5323-5326 but using HPLC grade CH₃CN (contains 0.5% water); no water was added.

### Step 10: N²-[(1S)-1-(4-bromophenyl)-2,2,2-trifluoroethyl]-N¹-(1-cyanocyclopropyl)-4-fluoro-L-leucinamide

Diisopropylethylamine (4.2 n3L) was added to a 0° C. suspension of the acid (1.5 g) from Step 9, 1-amino-1-cyclopropanecarbonitrile hydrochloride (1.18 g), O-(7-azabenzotriazol-1-yl)-N,N,N′,N′-tetramethyluronium hexafluorophosphate (1.94 g) and dimethylformamide (5 mL) and the mixture was reacted at room temperature for 48 hrs. It was then poured on ice and dilute aqueous ammonium chloride. The mixture was extracted with ethyl acetate and ether (1:1) and the combined organic layers were washed with pH 3 dilute Na₂HPO₄ and brine. The solvents were evaporated to dryness and the residue was purified by chromatography on SiO₂ using ethyl acetate and hexanes (1:2) to yield N²-[(1S)-1-(4-bromophenyl)-2,2,2-trifluoroethyl]-N¹-(1-cyanocyclopropyl)-4-fluoro-L-leucinamide in a sufficient purity state for the next step.

¹H NMR (CD₃COCD₃) δ 8.15(1H, NH), 7.6(2H, m), 7.45(2H, m), 4.35-4.45(1H, m), 3.45-3.55(1H, m), 1.9-2.1(2H, m), 1.75-1.85(1H, NH),1.35-1.55(8H, m), 1.1-1.15(1H, m), 0.95-1.05(1H, m).

### Step 11: N¹-(1-cyanocyclopropyl)-4-fluoro-N²-{(1S)-2,2,2-trifluoro-1-[4′-(methylthio)-1,1′-biphenyl-4-yl]ethyl}-L-leucinamide

A stream of nitrogen was passed through a suspension made of the bromide from Step 10 (0.338 g.), 4-(methylthio)phenylboronic acid (0.252 g), 2M Na₂CO₃ (0.8 mL) and DMF (4 mL) for 15 minutes. PdCl₂.dppf (0.1 g) was then added and the reaction was warmed to 85° C. and stirred under nitrogen for 5 hours. The mixture was cooled to room temperature, diluted with ethyl acetate (10 mL) and poured into water (50 mL) and ice. The ethyl acetate layer was separated and the aqueous further extracted with ethyl acetate. The combined ethyl acetate extracts were dried and the solvents removed in vacuo. The residue was purified by chromatography on SiO₂ using ethyl acetate and hexanes (1:2) to yield N¹-(1-cyanocyclopropyl)-4-fluoro-N²-{(1S)-2,2,2-trifluoro-1-[4′-(methylthio)-1,1′-biphenyl-4-yl]ethyl}-L-leucinamide.

¹H NMR (CD₃COCD₃) δ 8.15(1H, NH), 7.1-7.2(4H, m),7.5-7.55(2H, m), 7.35-7.4(2H, m), 4.3-4.4(1H, m), 3.45-3.55(1H, m), 2.75-2.8(1H, NH), 2.5(3H, s), 1.9-2.05(2H, m), 1.3-1.5(8H, m), 1.0-1.1(1H, m), 0.85-0.95(1H, m).

### Step 12: Preparation of N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide

To a 0° solution of the sulfide (0.265 g) from Step 11 in toluene (5 mL) and dichloromethane (5 mL) was added Na₂WO₄.2H₂O (0.002 g) and n-Bu₄NHSO₄ (0.01 g). Then 30% hydrogen peroxide (0.137 mL) was slowly added and the mixture was stirred at room temperature for 3 hours. The mixture was poured slowly on a mixture of ice, dilute aqueous sodium thiosulfate and ethyl acetate. The organic layer was separated and the aqueous further extracted with ethyl acetate. The combined organic layers were washed with brine, dried with magnesium sulfate and the solvent were removed in vacuo to yield a residue which was purified on SiO₂ using ethyl acetate, hexanes and dichloromethane (1:1:0.1) as eluant. The residue was triturated in diethyl ether to yield N¹-(1-cyanocyclopropyl)-4-fluoro-N²-{(1S)-2,2,2-trifluoro-1-[4′-(methylsulfonyl)-1,1′-biphenyl-4-yl]ethyl}-L-leucinamide

¹H NMR (CD₃COCD₃) δ 8.2(1H, NH), 8.05-8.1(2H, m), 7.95-8.0(2H, m), 7.8(2H, m), 7.65(2H, m), 4.35-4.45(1H, m), 3.5-3.6(1H, m), 3.2(3H, s), 2.8-2.9(1H, NH), 1.9-2.1(2H, m), 1.3-1.5(8H, m), 1.05-1.15(1H, m), 0.9-1.0(1H, m).

### EXAMPLE 6

### Synthesis of N²-[1-(3-bromophenyl)-2,2,2-trifluoroethyl]-N¹-(cyanomethyl)-L-leucinamide

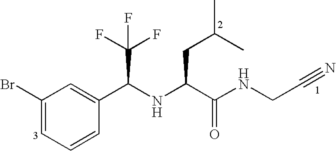

Using the procedure described for Example 1, where 2,2,2-trifluoroacetophenone was substituted for 1-(3-bromophenyl)-2,2,2-trifluoroethanone, the title compound was prepared.

MS (+ESI): 406.0, 408.1 [M+1]⁺.

### EXAMPLE 7

### N¹-(Cyanomethyl)-N²-[2,2,2-trifluoro-1-(4-piperazine-1-ylphenyl)ethyl]-L-leucinamide

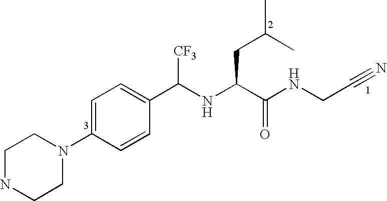

### Step 1: N²-(1-{4-[4-(tert-butylcarboxylate)piperazin-1-yl[phenyl}-2,2,2-trifluoroethyl)-N¹-(cyanomethyl)-L-leucinamide

A solution of N²-[1-(4-bromophenyl)-2,2,2-trifluoroethyl]-N1-(cyanomethyl)-L-leucinamide (example 2) (100 mg, 0.25 mmol), tris(dibenzylideneacetone)dipalladium (2.3mg, 0.0025 mmol), biphenyl-2-yl(di-tert-butyl)phosphine (3 mg, 0.01 mmol), potassium phosphate (74 mg, 0.35 mmol) and tert-butyl piperazine-1-carboxylate (56 mg, 0.3 mmol) in dimethoxyethane (0.5 mL) was cooled to −78° C. pumped under high vacuum for 3 minutes, then nitrogen was admitted in the flask. The mixture was then heated at 80° C. for 1 hr. After cooling to room temperature, the mixture was applied directly on a silica gel column and eluted with ethyl acetate/ hexane to afford the title compound.

### Step 2: N¹-(Cyanomethyl)-N²-[2,2,2-trifluoro-1-(4-piperazin-1-ylphenyl)ethyl]-L-leucinamide

To the compound from step 1 ( 139 mg, 0.27 mmol) in 1,4-dioxane (0.5 mL) was added methanesulfonic acid (53 uL, 0.82 mmol) and the mixture was stirred over night. Ethyl acetate was added, then the mixture was washed with saturated sodium bicarbonate, brine, dried over magnesium sulfate, filtered and the solvent evaporated under vacuum. Purification by silica gel chromatography eluting with 93% dichloromethane, 0.6% ammonium hydroxide and 6.4% methanol afforded the title compound.

MS (+ESI): 412.2 [M+1]⁺.

### EXAMPLE 8

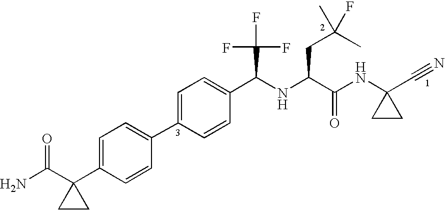

### Synthesis of N²-((1S)-1-{4′-[1-(aminocarbonyl)cyclopropyl]biphenyl-4-yl}-2,2,2-trifluoroethyl)-N¹-(1-cyanocyclopropyl)-4-fluoro-L-leucinamide

### Step 1: Preparation of 1-(4-bromophenyl)cyclopropanecarbonitrile

To a room temperature solution of 4-bromophenylacetonitrile (18.0 g) in 22 mL of sodium hydroxide (50% in water W/W) were added 1-bromo-2-chloroethane and (12.0 mL) and benzyltrimethylammonium chloride (627 mg). The mixture was heated at 60° C. overnight. The reaction mixture was cooled to room temperature and diethyl ether was added (300 mL. The ether layer was washed with water (100 mL), hydrogen chloride (100 mL, 10% HCl in water) and brine. The organic layer was dried with magnesium sulfate and the solvent removed in vacuo. The residue was purified by trituration using diethyl ether and hexanes to yield the title compound.

¹H NMR (CD₃COCD₃) δ 7.60(2H, d), 7.35(2H, d), 1.74-1.80(2H, m), 1.52-1.57(2H, m).

### Step 2: Preparation of 1-(4-bromophenyl)cyclopropanecarboxylic acid

To a room temperature solution of 1-(4-bromophenyl)cyclopropanecarbonitrile from Step 1 (13 g) in ethyl alcohol (110 mL) was added a solution of 56 mL of sodium hydroxide (25% NaOH in water W/W). The mixture was heated at 100° C. overnight. It was cooled to room temperature, poured into ice and hydrogen chloride (1 N) and extracted with dichloromethane (2×100 mL). The combined extracts were washed with brine, dried with magnesium sulfate and the solvent removed in vacuo to yield the title compound.

¹H NMR (CD₃COCD₃) δ 7.50(2H, d), 7.35(2H, d), 1.53-1.60(2H, m), 1.18-1.22(2H, m).

### Step 3: Preparation of 1-(4-bromophenyl)cyclopropanecarboxamide

To a −15° C. solution of 1-(4-bromophenyl)cyclopropanecarboxylic acid from Step 2 (1.5 g) in chloroform (60 mL) were slowly added isobutyl chloroformate (900 μL) and triethylamine (1.1 mL). The reaction mixture was stirred at −15° C. for 2 hours. Then it was saturated with ammonia gas and stirred at −15° C. for 10 minutes. The reaction mixture was allowed to stand at room temperature for 1 hour then poured into water (60 mL) and partitioned. The aqueous layer was extracted with dichloromethane (2×60 mL). The combined extracts were washed with brine, dried with magnesium sulfate and the solvent removed in vacuo. The residue was purified by swish using diethyl ether and hexanes to yield the tide compound.

¹H NMR (CD₃COCD₃) δ 7.54(2H, d), 7.40(2H, d), 6.45(1H, bs), 5.96(1H, bs), 1.42-1.48(2H, m), 0.98-1.02(2H, m).

### Step 4: Preparation of N¹-(1-cyanocyclopropyl)-4-fluoro-N²-{(1S)-2,2,2-trifluoro-1-[4-(4,4,5,5-tetramethyl-1,3,2-dioxaborolan-2-yl)phenyl]ethyl}-L-leucinamide

A stream of nitrogen was passed through a DMF (40 mL) suspension of N²-[(1S)-1-(4-bromophenyl)-2,2,2-trifluoroethyl]-N¹-(1-cyanocyclopropyl)-4-fluoro-L-leucinamide from Example 5, Step 9 (2.0 g), bis(pinacolato)diboron (1.24 g) and potassium acetate (1.53 g) for 15 minutes. The catalyst [1,1′-bis(diphenylphosphino)-ferrocene]dichloropalladium(I), complex (1:1) with dichloromethane (181 mg) was then added and the mixture warmed to 65° C. overnight under nitrogen. The mixture was cooled to room temperature, diluted with ethyl acetate and hexanes (1: 1, 100 mL) and poured over water (50 mL) and ice (50 g). The organic layer was separated and the aqueous layer further extracted with ethyl acetate and hexanes (1:1, 3×50 mL). The combined extracts were washed with brine and dried with magnesium sulfate. Removal of the solvent left a residue which was purified by chromatography on SiO₂ using ethyl acetate and hexanes (1:3 to 1:2) to yield the title compound.

¹H NMR (CD₃COCD₃) δ 8.15(1H, bs), 7.78(2H, d), 7.50(2H, d), 4.31-4.40 (1H, m), 3.47-3.54 (1H, m), 2.72-2.80 (2H, m), 1.32-1.48(9H, m), 1.05-1.11(1H, m), 0.87-0.95(1H, m).

### Step 5: Preparation of N²-((1S)-1-{4′-[1-(aminocarbonyl)cyclopropyl]biphenyl-4-yl}-2,2,2-trifluoroethyl)-N¹-(1-cyanocyclopropyl)-4-fluoro-L-leucinamide

A stream of nitrogen was passed through a solution of DMF (4 mL) of the boronate from Step 4 (150 mg), 1-(4-bromophenyl)cyclopropanecarboxamide from Step 3 (110 mg) and 2 M Na₂CO₃ (400 μL) for 15 minutes. The catalyst [1,1′-bis(diphenylphosphino)-ferrocene]dichloropalladium(II), complex (1:1) with dichloromethane (12 mg) was then added and the mixture was warmed to 80° C. for 3 hours under nitrogen. The mixture was cooled to room temperature, poured into ice (10 g) and saturated aqueous sodium bicarbonate (20 mL) and extracted with 50% ethyl acetate (3×30 mL). The combined extracts were washed with brine and dried with magnesium sulfate. Removal of the solvent left a residue which was purified by chromatography on SiO₂ using ethyl acetate and hexanes (50 to 70%) as eluants, followed by a swish using diethyl ether to yield the title compound.

¹H NMR (CD₃COCD₃) δ 8.20(1H, bs), 7.75(2H, d), 7.70(2H, d), 7.60(2H, d), 7.55(2H, d), 6.37(1H, bs), 5.87(1H, bs), 4.35-4.43 (1H, m), 3.52-3.58 (1H, m), 1.92-2.05 (2H, m), 1.42-1.50(6H, m), 1.35-1.42(4H, m), 1.03-1.12(3H, m), 0.92-0.98(1H, m).

### EXAMPLE 9

### N²-[(1S)-1-(4-bromophenyl)-2,2,2-trifluoroethyl]-N¹-(1-cyanocyclopropyl)-4,4-difluoro-L-norvalinamide

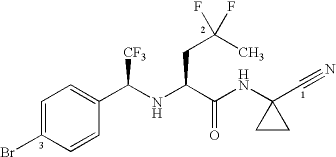

### Step 1: Preparation of methyl N-((benzyloxy)carbonyl)-3-iodo-L-alanimate

To a solution of carbobenzyloxy-L-serine (25 g, 104 mmol) in ethyl acetate (200 mL) was added a solution of diazomethane in ether until a slight yellow color persisted. The solvent was evaporated under vacuum To the residue was added N,N-dimethylformamide (400 mL) and methyltriphenoxyphosphonium iodide (50 g, 110 mmol). The mixture was stirred for 15 minutes, then methanol (15 mL) was added and the mixture was then poured over 20% sodium thiosulfate and extracted with a 1:1 mixture of ethyl acetate:hexanes (2 L). The organic layer was washed with water, brine (3×), dried over magnesium sulfate, filtered and the solvent evaporated under vacuum. The residue was purified by silica gel chromatography using ethyl acetate and hexanes. The compound obtained was triturated in diethyl ether/hexanes, filtered and air dried to afford methyl N-((benzyloxy)carbonyl)-3-iodo-L-alaninate.

### Step 2: Preparation of methyl N-(benzyloxy)carbonyl)-4-oxo-L-norvalinate

A mixture of methyl N-((benzyloxy)carbonyl)-3-iodo-L-alaninate (10 g, 27.5 mmol), from Step 1, zinc-copper couple (3.3 g) in benzene (110 mL) and N,N-dimethylacetamide (7.4 mL) was sonicated in an ultra-sound bath for 2 hours. Over this period, 3 portions of 1,2-dibromoethane (0.24 mL) and chlorotrimethylsilane (0.17 mL) were added. To this mixture was then added bis(triphenylphosphine)palladium chloride (0.958 g, 1.4 mmol) and acetyl chloride (2.5 mL, 35.2 mmol) and the mixture was heated at 70° C. for 2 hours. After cooling to room temperature, the mixture was filtered on celite with ethyl acetate, the organic layer was then washed with a saturated solution of ammonium chloride, brine (2×), dried over magnesium sulfate, filtered and the solvent evaporated under vacuum. The residue was purified by silica gel chromatography using ethyl acetate and hexanes to afford methyl N-((benzyloxy)carbonyl)-4-oxo-L-norvalinate.

### Step 3: Preparation of methyl N-((benzyloxy)carbonyl)-4,4-difluoro-L-norvalinate

To a solution of methyl N-((benzyloxy)carbonyl)4-oxo-L-norvalinate (1.3 g, 4.65 mmol) in dichloromethane (20 mL) and methanol (0.019 mL) at 0° C. was added DAST (2.46 mL) slowly. The ice bath was removed and replaced with a hot water (57° C.) bath. The hot water bath was replaced 3 times, then the mixture was stirred overnight at room temperature. The mixture was slowly poured over cold saturated NaHCO₃, extracted with ethyl acetate, washed with brine, dried over magnesium sulfate, filtered and the solvent evaporated under vacuum. The residue was purified by silica gel chromatography using ethyl acetate and hexanes to afford methyl N-((benzyloxy)carbonyl)-4,4-difluoro-L-norvalinate.

### Step 4: Preparation of benzyl (1S)-3,3-difluoro-1-(hydroxymethyl)butylcarbamate

To a solution of methyl N-((benzyloxy)carbonyl)-4,4-difluoro-L-norvalinate (1.59 g, 5.29 mmol) in ethanol (50 mL) was added lithium chloride (919 mg) and the mixture was stirred for 10 minutes. Sodium borohydride (820 mg) was added slowly, the mixture stirred for 2 hours. Then, another portion of sodium borohydride (100 mg) was added and stirring continued for 30 minutes. The mixture was diluted with water (20 mL) and neutralized slowly with 1N HCl followed by the addition of another aliquot of water. The mixture was extracted with ethyl acetate (2×), washed with brine, dried over magnesium sulfate, filtered and the solvent evaporated under vacuum to afford benzyl (1S)-3,3-difluoro-1-(hydroxymethyl)butylcarbamate.

### Step 5: Preparation of (2S)-1-((tert-butyl(dimethyl)silyl)oxy)-4,4-difluoropentan-2-amine

To a solution of benzyl (1S)-3,3-difluoro-1-(hydroxymethyl)butylcarbamate (from Step 4) in ethanol (25 mL) was added palladium on charcoal (10%, 150 mg) and the mixture was stirred under a H₂ atmosphere (ballon) for 2 h. Dichloromethane was added and the mixture was filtered on celite. The solvent was evaporated under vacuum. The residue was dissolved in dichloromethane (15 mL) and triethylamine (1 mL), N,N-dimethylaminopyridine (10 mg) and chloro-t-butyldimethylsilane (844 mg) were added. The mixture was stirred overnight, then water and brine were added. The mixture was extracted with ethyl acetate (2×), washed with brine, dried over magnesium sulfate, filtered and the solvent evaporated under vacuum to afford (2S)-1-((tert-butyl(dimethyl)silyl)oxy)-4,4-difluoropentan-2-amine.

### Step 6: Preparation of (2S)-1-((tert-butyl(dimethyl)silyloxy)-4,4-difluoro-N-((1E)-2,2,2-trifluoroethylidene)pentan-2-amine

A solution of (2S)-1-((tert-butyl(dimethyl)silyl)oxy)-4,4-difluoropentan-2-amine, from Step 5, and trifluoroacetaldehyde methyl hemiacetal (80%, 0.9 mL) in benzene (20 mL) was refluxed over night with a Dean-Stark apparatus. The solvent was evaporated under vacuum and the residue purified by silica gel chromatography using ethyl acetate and hexanes to afford (2S)-1-((tert-butyl(dimethyl)silyl)oxy)-4,4-difluoro-N-((1E)-2,2,2-trifluoroethyldene)pentan-2-amine.

### Step 7: Preparation of (2S)-2-(((1S)-1-(4-bromophenyl)-2,2,2-trifluoroethyl)amino)-4,4-difluoropentan-1-ol

To a −78° C. solution of 1,4-dibromobenzene (330 mg) in THF (5.2 mL) was added 2.5M n-BuLi in hexanes (0.52 mL) and the solution was aged for 30 minutes. Then, a solution of (2S)-1-((tert-butyl(dimethyl)silyl)oxy)-4,4-difluoro-N-((1E)-2,2,2-trifluoroethy(idene)pentan-2-amine (333 mg) in TBF (5.2 mL) was added. The mixture was stirred at −78° C. for 45 minutes, then poured over cold saturated ammonium chloride, extracted with ethyl acetate (2×), washed with brine, dried over magnesium sulfate, filtered and the solvent evaporated under vacuum. The residue was dissolved in THF (10 mL) cooled in an ice/water bath and n-tetrabutylammonium fluroride (1M in THF, 1.5 mL) was added. The mixture was stirred at 0° C. for 1 h, poured over cold water, extracted with ethyl acetate (2×), washed with brine, dried over magnesium sulfate, filtered and the solvent evaporated under vacuum to afford (2S)-2-(((S)-1-(4-bromophenyl)-2,2,2-trifluoroethyl)amino)4,4-difluoropentan-1-ol.

### Step 8: Preparation of N²-[(1S)-1-(4-bromophenyl)-2,2,2-trifluoroethyl]-N¹-(1-cyanocyclopropyl)-4,4-difluoro-L-norvalinamide

A suspension of H₅IO₆ /CrO₃ (27 mL of 0.44 M in CH₃CN; Note) was cooled to 0° C. and a solution of the alcohol from Step 7 (740 mg) in CH₃CN (10 mL) was added dropwise. The mixture was stirred at 0° C. for 4 hours, with addition of more H₅IO₆ /CrO₃ (2×10 mL of 0.44 M in CH₃CN). Then the mixture was poured into pH 4 Na₂HPO₄ buffer under vigorous stirring and the mixture was extracted with ethyl acetate washed with brine (2×) followed by dilute aqueous NaHSO₃ and brine. The mixture was dried with magnesium sulfate, filtered and the solvents were evaporated to dryness to yield 680 mg of N-[(1S)-1-(4-bromophenyl)-2,2,2-trifluoroethyl]-4,4-difluoro-L-norvaline which was used as such in the next step.

Note. The oxidizing reagent (H₅IO₆ /CrO₃) was prepared as described in Tetrahedron Letters 39 (1998) 5323-5326 but using HPLC grade CH₃CN (contains 0.5% water); no water was added.

Triethylamine (0.42 mL) was added to mixture of the acid (340 mg) from above, 1-amino-1-cyclopropanecarbonitrile hydrochloride (227 mg), benzotriazol-1-yl-oxytripyrrolidinophosphonium hexafluorophosphate (498 mg) and dimethylformamide (4.5 mL) and the mixture was reacted at room temperature for 48 h. It was then poured on dilute sodium bicarbonate. The mixture was extracted with ethyl ether (3×) and the combined organic layers were washed with brine (3×) and dried with magnesium sulfate filtered. The solvents were evaporated to dryness and the residue was purified by chromatography on silica gel using ethyl acetate 40% and hexanes to yield N²-[(1S)-1-(4-bromophenyl)-2,2,2-trifluoroethyl]-N¹-(1-cyanocyclopropyl)-4,4-difluoro-L-norvalinamide.

MS (+ESI): 454.1, 456.2 [M+1]⁺.

### EXAMPLE 10

### N²-[(1S)-1-(4-bromophenyl)-2,2,2-trifluoroethyl]-N¹-(cyanomethyl)-4,4-difluoro-L-norvalinamide

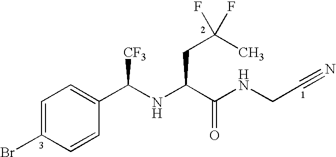

Using the procedure described for Example 9, where 1-amino-1-cyclopropanecarbonitrile hydrochloride was substituted for aminoacetonitrile hydrochloride in Step 8, the title compound was obtained

MS (+ESI): 428, 430.1 [M+1]⁺.

### EXAMPLE 11

### 4-fluoro-N¹-[(2R,3S)-2-methyl-4-oxotetrahydrofuran-3-yl]-N²-{(1S)-2,2,2-trifluoro-1-[4′-(methylsulfonyl)biphenyl-4-yl]ethyl}-L-leucinamide

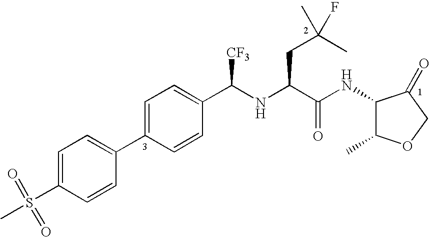

### Step 1: N²-[(1S)-1-(4-bromophenyl)-2,2,2-trifluoroethyl]-fluoro-N¹-[(2R,3S)-2-methyl-4-oxotetrahydrofuran-3-yl]-L-leucinamide

Triethylamine (0.63 mL) was added to mixture of the acid (500 mg) from Example 5, step 9, (4S,5R)-4-amino-5-methyldihydrofuran-3-(2H)-one hydrochloride (227 mg) (WO 00169855), benzotriazol-1-yl-oxytripyrrolidinophosphonium hexafluorophosphate (744 mg) and dimethylformamide (7 mL) and the mixture was reacted at room temperature for 3 hrs. It was then poured on dilute sodium bicarbonate. The mixture was extracted with ethyl ether (3×) and the combined organic layers were washed with brine (3×) and dried with magnesium sulfate filtered. The solvents were evaporated to dryness and the residue was purified by chromatography on silica gel using ethyl acetate 30% and hexanes to yield the title compound.

### Step 2: 4-fluoro-N¹-[(2R,3S)-2-methyl-4-oxotetrahydrofuran-3-yl]-N²-{(1S)-2,2,2-trifluoro-1-[4′-(methylthio)biphenyl-4-yl]ethyl}-L-leucinamide

A mixture of the bromide from Step 1 (200 mg), 4-(methylthio)phenylboronic acid (104 mg), 2M aqueous Na₂CO₃ (0.51 mL) and DMF (3 mL) for 15 minutes. PdCl₂dppf₂ (17 mg) was cooled to −78° C., pumped under high vacuum for 5 minutes, then nitrogen was let into the flask and the mixture was heated at 80° C. and stirred under nitrogen for 3 hours. The mixture was cooled to room temperature, diluted with ethyl acetate washed with saturated ammonium chloride, brine (3×), dried over magnesium sulfate, filtered and the solvent evaporated under vacuum. Purification by silica gel chromatography using 35% ethyl acetate/hexane as eluent afforded the title compound.

### Step 3: 4-fluoro-N¹-[(2R,3S)-2-methyl-4-oxotetrahydrofuran-3-yl]-N²-{(1S)-2,2,2-trifluoro-1-[4′-(methylsulfonyl)biphenyl-4-yl]ethyl}-L-leucinamide

To a solution of the sulfide (120 mg) from Step 2 in ethyl acetate (2 mL) was added Na₂WO₄.2H₂O (1 mg) and n-Bu₄NHSO₄ (4 mg). Then 30% hydrogen peroxide (0.06 mL) was slowly added and the mixture was stirred at room temperature for 2 hours. More hydrogen peroxide was then added (0.06 mL). Ethyl acetate was then added to the mixture which was washed with concentrated aqueous Na₂S₂O₃ (2×), brine, dried over magnesium sulfate, filtered and the solvent evaporated under vacuum. Purification by silica gel chromatography using 55% ethyl acetate/hexane as eluent afforded the title compound.

MS (+ESI): 559.1 [M+1]⁺.

### EXAMPLE 12

### Synthesis of N¹-(1-cyano-1-methylethyl)-N²-{(1S)-2,2,2-trifluoro-1-[4′-(methylsulfonylbiphenyl-4-yl]ethyl}-L-leucinamide

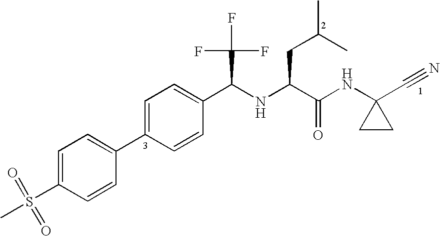

### Step 1: Preparation of 2-amino-2-methylpropanenitrile hydrochloride

To a 0° C. solution of ammonium chloride (15.5 g) in water (50 mL) was added a solution of acetone (17 mL) in diethyl ether (50 mL). Then a solution of the sodium cyanide (11.9 g.) in water (35 mL) was slowly added at such a rate that the temperature never exceeds 10° C. The reaction mixture was stirred for one hour at 0° C. after the addition of the cyanide solution then it was allowed to stand overnight. The ether layer was separated and the aqueous layer was extracted with diethyl ether (2×30 mL). The combined organic layers were washed with brine, dried with magnesium sulfate and the solvent were removed in vacuo to yield a residue which was diluted with methyl alcohol (80 mL). The solution was cooled at −78° C. and saturated with ammonia gas (An Ace pressure tube with plunger valve and thermo well was used). The reaction mixture was allowed to stand at room temperature for two days. The excess ammonia was expelled by a current of air and the methyl alcohol was removed by evaporation. The residue was dissolved in diethyl ether (50 mL) and cooled to 0° C. then a solution of 40 mL of hydrogen chloride (1.0 M in diethyl ether) was added. The mixture was stirred for 30 minutes and filtered to yield the title compound.

¹H NMR (CD₃SOCD₃) δ 9.45(1H, s), 1.70(6H, s).

### Step 2: Preparation of N¹-(1-cyano-1-methylethyl)-N²-{(1S)-2,2,2-trifluoro-1-{4′-(methylsulfonyl)biphenyl-4-yl]ethyl}-L-leucinamide

To a DMF (30 mL) solution of the acid from Example 4, Step 6 (1.2 g) was added benzotriazol-1-yloxytris(dimethylamino)phosphoniumhexafluorophosphate (1.55 g), 2-amino-2-methylpropanenitrile hydrochloride from Step 1 (720 mg) and the mixture was cooled to 0° C. Triethylamine (1.3 mL) was added drop wise and the mixture was warmed to room temperature and stirred for 72 hours. It was poured into ice and saturated aqueous sodium bicarbonate and extracted with diethyl ether (3×50 mL). The combined extracts were washed with brine, dried with magnesium sulfate and the solvent removed in vacuo. The residue was purified by chromatography on SiO₂ using a gradient of ethyl acetate and hexanes (1:2 to 1:1) as eluant, followed by trituration using diethyl ether and hexanes to yield the title compound.

¹H NMR (CD₃COCD₃) δ 8.08(2H, d), 7.95(2H, d), 7.80(2H, d), 7.68(1H, bs), 7.65(2H, d), 4.32-4.42 (1H, m), 3.43-3.52 (1H, m), 3.20 (3H, s), 2.65-2.75(1H, m), 1.90-2.00(1H, m), 1.57(3H, s), 1.52(3H, s), 1.52-1.57 (1H, m), 1.40-1.50(1H, m), 0.90-0.98(6H, m).

### EXAMPLE 13

### N¹-(Cyanomethyl)-3-(1-methylcyclopropyl)-N²-{2,2,2-trifluoro-1-[4′-(methylsulfonyl)-1,1′-biphenyl-4-yl]ethyl}-alaninamide

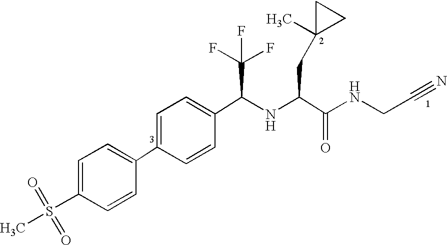

### Step 1: Methyl N-(diphenylmethylene)-4-methylenenorvalinate

To a solution of methyl N-(diphenylmethylene)glycinate (12.0 g, 47.4 mmol) in THF (118 mL) at 0° C. was added a solution of 1M potassium tert-butoxide in TBF (49 mL, 49 mmol). The mixture turned bright yellow and was further stirred for ˜15 min. 3-Bromo-2-methylpropene (5.2 mL, 51.3 mmol) was added and the mixture was stirred at room temperature for 2 days. After quenching with water, the mixture was extracted with EtOAc. Chromatography over silica gel and elution with hexanes:EtOAc (6:1) provided 2.2 g of dialkylated product as the less polar component. Further elution gave the title compound as the more polar component.

¹H NMR (Acetone-d₆) δ 7.62-7.18 (m, 10H), 4.72 (s, 1H), 4.64 (s, 1H), 4.20 (dd, 1H), 3.64 (s, 3H), 2.62 (dd, 1H), 2.50 (dd, 1H), 1.48 (s, 3H).

### Step 2: Methyl N-[(benzyloxy)carbonyl]-methylenenorvalinate

A mixture of methyl N-(diphenylmethylene)-4-methylenenorvalinate (6.2 g, 20.2 mmol) from Step 1 and 0.5 M of aqueous HCl (60 mL) was stirred at room temperature overnight. The whole mixture was washed with Et₂O (2×). After cooling to 0° C., 1M aqueous NaOH (40 mL, 40 mmol) was added, followed by EtOAc (50 mL) and benzyl chloroformate (4 mL, 28 mmol). The mixture was stirred at 0° C. for 2 h. The EtOAc layer was then separated, washed with water (2×), dried (MgSO₄) and concentrated. Chromatography over silica gel and elution with hexanes:EtOAc (6:1), then (3:1) gave the title compound as a colorless oil.

¹H NMR (Acetone-d₆) δ 7.40-7.25 (m, 5H), 6.54 (d, 1H), 5.06 (s, 2H), 4.82 (s, 1H), 4.78 (s, 1H), 4.40 (m, 1H), 3.68 (s, 3H), 2.52 (dd, 1H), 2.40 (dd, 1H), 1.74 (s, 3H).

### Step 3: Methyl N-[(benzyloxy)carbonyl]-3-(1-methylcyclopropyl)alaninate

To CH₂Cl₂ (40 mL) at 0° C. was added diethylzinc (2 mL, 19.5 mmol), followed by dropwise addition of a solution of trifluoroacetic acid (1.5 mL, 19.5 mmol) in CH₂Cl₂ (8 mL). After stirring for 15 min, a solution of diiodomethane (1.6 mL, 20.0 mmol) in CH₂Cl₂ (8 mL) was added. The mixture was stirred for 15 min and a clear solution resulted. A solution of methyl N-[(benzyloxy)carbonyl]-4-methylenenorvalinate (2.75 g, 9.9 mmol) from step 2 was added and the mixture was stirred at room temperature overnight. After quenching with 0.1 M aqueous HCl (50 mL), the CH₂Cl₂ layer was separated, washed with diluted brine, dried (MgSO₄) and concentrated to give the crude tilte compound.

¹H NMR (Acetone-d₆) δ 7.35 (m, 5H), 6.58 (d, 1H), 5.08 (m, 2H), 4.40 (m, 1H), 3.68 (s, 3H), 1.75 (dd, 1H), 1.62 (dd, 1H), 1.08 (s, 3H), 0.45 (m, 1H), 0.22 (m, 3H).

### Step 4: Benzyl 2-hydroxy-1-[(1-methylcyclopropyl)methyl]ethylcarbamate

To a solution of the crude ester from step 3 in EtOH (40 mL) and THF (40 mL) and cooled at 0° C. was added LiCl (1.7 g), followed by NaBH₄ (1.6 g). The mixture was stirred at room temperature overnight, quenched with 0.5 M aqueous HCl, extracted with EtOAc. The EtOAc extract was washed with diluted brine (2×), dried (MgSO₄) and concentrated. Purification by chromatography gave 2 g of the title compound as a colorless oil.

¹H NMR (Acetone-d₆) δ 7.35 (m, 5H), 5.98 (d, 1H), 5.06 (m, 2H), 3.85 (m, 1H), 3.72 (t, 1H), 3.50 (m, 2H), 1.60 (dd, 1H), 1.34 (dd, 1H), 1.05 (s, 3H), 0.40-0.15 (m, 4H).

### Step 5: 2-Amino-3-(1-methylcyclopropyl)propan-1-ol

A mixture of benzyl 2-hydroxy-1-[(1-methylcyclopropyl)methyl]-ethylcarbamate (2.0 g, 7.6 mmol) and 10% Pd/C (200 mg) in EtOH (80 mL) with 2 mL of 1 M aqueous HCl was stirred H₂ atmosphere (balloon) overnight. The catalyst was filtered off and the filtrate was concentrated to give the title compound.

¹H NMR (Methanol-d₄) δ 3.72 (dd, 1H), 3.40 (dd, 1H), 3.22 (m, 1H), 1.50-1.30 (m, 2H), 1.06 (s, 3H), 0.45-0.25 (m, 4H).

### Step 6: N¹-(Cyanomethyl)-3-(1-methylcyclopropyl)-N²-{2,2,2-trifluoro-1-[4′-(methylsulfonyl)-1,1′-biphenyl-4-4-yl]ethyl }-alaninamide

The title compound was prepared from the aminoalcohol from Step 5 using the same method described in Example 5, Steps 6-12.

¹H NMR (Acetone-d₆) δ 8.06 (br s, 1H), 8.00 (d, 2H), 7.94 (d, 2H), 7.78 (d, 2H), 7.62 (d, 2H), 4.48 (m, 1H), 4.12 (m, 2H), 3.55 (m, 1H), 3.16 (s, 3H), 1.64 (m, 2H), 1.10 (s, 3H), 0.48-0.20 (m, 4H).

MS (+ESI): 494 (MH⁺).

### EXAMPLE 14

### Synthesis of N¹-(cyanomethyl)-N²-(2,2,2-trifluoro-1-{4-[(4-methylpiperazin-1-yl)carbonyl]phenyl}ethyl)-L-leucinamide

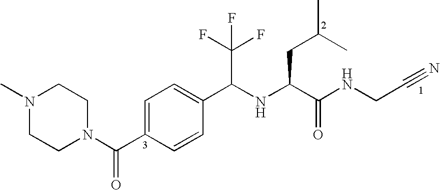

### Step 1: N²-{1-[4-(hydroxycarbonyl)phenyl]-2,2,2-trifluoroethyl}-N-(cyanomethyl)-L-leucinamide

A mixture of dichlorobis(triphenylphosphine)palladium(II) (58 mg, 0.08 mmol), triphenylphosphine (155 mg, 0.59 mmol) and N²-[1-(4-bromophenyl)-2,2,2-trifluoroethyl]-N¹-(cyanomethyl)-L-leucinamide (Example 2, 1.2 g, 3.0 mmol) in tributylamine (2.5 mL) and water (0.6 mL) was placed in a steel bomb with a teflon coated magnetic bar. The system was purged 3 times with carbon monoxide (100 psi each times) and finally filled with this gas at a pressure of 300 psi. The reaction mixture was heated with continuous stirring at 160° C. for 20 hours. Then the system was allowed to cool to room temperature, pressure was released and the resulting residue was partitioned between EtOAc and water+aqueous hydrochloric acid to adjust the pH between 2.5 and 3.0. The organic layer was dried over Na2SO4, filtered and concentrated. The crude product was purified by chromatography using EtOAc, hexane and acetic acid as eluant to give the title compound as a yellow-orange foam.

### Step 2: N¹-(cyanomethyl)-N²-(2,2,2-trifluoro-1-{4-[(4-methylpiperazin-1-yl)carbonyl]phenyl}ethyl)-L-leucinamide

To a solution of N²-{1-[4-(hydroxycarbonyl)phenyl]-2,2,2-trifluoroethyl}-N¹-(cyanomethyl)-L-leucinamide from step 1 (480 mg, 1.3 mmol) and benzotriazol-1-yloxytrpyrrolidinophosphonium hexafluorophosphonate (1.35 g, 2.6 mmol) in DMF (8 mL) was slowly added N-methylpiperazine (0.29 mL, 2.6 mmol) followed by triethylamine (0.54 mL, 3.9 mmol). The reaction mixture was stirred at room temperature for 3 hours. The resulting mixture was partitioned between EtOAc and water+aqueous hydrochloric acid to adjust the pH between 2.5 and 3.0. The organic layer was dried over Na₂SO₄, filtered and concentrated. The crude product was purified by chromatography using MeOH, EtOAc and NH₄OH_{conc} as eluant to give the title compound as a white foam.

MS (+ESI): 454.3 [M+1]⁺.

### EXAMPLE 15

### Synthesis of N¹-{(1S)-1-[2-(methylthio)ethyl]-2-oxopropyl}-N²-{(1S)-2,2,2-trifluoro-1-[4′-(methylsulfonyl)biphenyl-4-yl]ethyl}-L-leucinamide

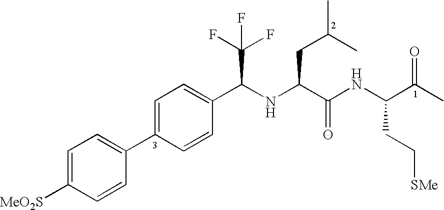

### Step 1: N²-(tert-Butoxycarbonyl)-N¹-methoxy-N-methyl-L-methioninamide

To an ice-cold solution of N-Boc-L-methionine (1.0 g, 4.0 mmol), O-(7-azabenzotriazol-1-yl)-N,N,N′,N′-tetramethyluronium hexafluorophosphate (3.3 g, 8.7 mmol) and N,O-dimethylhydroxylamine hydrochloride (1.0 g, 10.2 mmol) in DMF (15 mL) was added triethylamine (2.2 mL, 15.8 mmol) dropwise. The resulting mixture was stirred at room temperature for 18 hours and then partitioned between EtOAc and half-saturated aqueous NaHCO₃. The organic layer was dried over Na₂SO₄, filtered and concentrated. The crude product was purified by chromatography using EtOAc and hexane as eluant to give the title compound as a colorless syrup.

### Step 2: tert-Butyl{(1S)-1-[2-(methylthio)ethyl]-2-oxopropyl}carbamate

To a solution of N²-(tert-butoxycarbonyl)-N¹-methoxy- N¹-methyl-L-methioninamide from Step 1 (200 mg, 0.68 mmol) in THF cooled to −78° C. was added a solution of methyl lithium 1.4 M in hexane (1.1 mL, 1.5 mmol). The reaction was stirred at this temperature for 2 hours and then cold-quenched with an aqueous solution of ammonium acetate 25% W/v. The mixture was extracted with EtOAc, the organic layer was dried over Na₂SO₄, filtered and concentrated. The crude product was purified by chromatography using EtOAc and hexane as eluant to give the title compound as a colorless gum.

### Step 3: (3S)-3-Amino-5-(methylthio)pentan-2-one, hydrochloride

To tert-butyl {(1S)-1-[2-(methylthio)ethyl]-2-oxopropyl}carbamate from step 2 (140 mg, 0.57 mmol) was added a solution hydrogen chloride 4.0 M in 1,4-dioxane (3 mL, 12 mmol). The resulting mixture was stirred at room temperature for 1 hour. The solvent was removed in vacuo and the resulting residue azeotroped with toluene (2×10 mL) to give the title compound as a white solid.

### Step 4: N¹-{(1S)-1-[2-(methylthio)ethyl]-2-oxopropyl}-N²-{(1S)-2,2,2-trifluoro-1-[4′-(methylsulfonyl)biphenyl-4-yl]ethyl}-L-leucinamide

A suspension of (3S)-3-amino-5-(methylthio)pentan-2-one, hydrochloride from Step 3 (104 mg, 0.57 mmol), N-{(1S)-2,2,2-trifluoro-1-[4′-(methylsulfonyl)-1,1′-biphenyl-4-yl]ethyl}-L-leucine (Example 4, Step 6, 127 mg, 0.2 mmol) and O-(7-azabenzotriazol-1-yl)-N,N,N′,N′-tetramethyluronium hexafluorophosphate (187 mg, 0.49 mmol) in DMF (1 mL) was cooled to 10° C. and then triethylamine (115 μL, 0.83 mmol) was added slowly . The reaction mixture was stirred at room temperature for 3 hours. The resulting mixture was partitioned between EtOAc and half-saturated aqueous NaHCO₃. The organic layer was dried over Na₂SO₄, filtered and concentrated. The crude product was purified by chromatography using EtOAc and hexane as eluant to give the title compound as a white foam.

MS (+ESI): 573.5 [M+1]⁺.

### EXAMPLE 16

### Synthesis of N¹-{(1S)-2,2,2-trifluoro-1-[4′-(methylsulfonyl)biphenyl-4-yl]ethyl}-L-leucyl-L-methioninamide

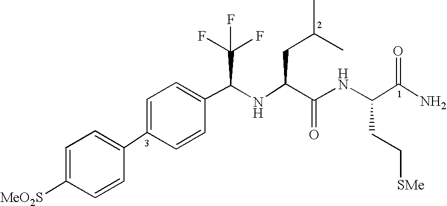

Using the procedure described for Example 15, Step 4, where (3S)-3-amino-5-(methylthio)pentan-2-one, hydrochloride was substituted for L-methioninamide hydrochloride, the title compound was obtained and was crystallized from EtOAc and hexane (1:2) to give a white solid.

MS (+ESI): 574.3 [M+1]⁺.

### EXAMPLE 17

### Synthesis of N-{(1S)-2,2,2-trifluoro-1-[4-(methylsulfonyl)biphenyl-4-yl]-ethyl}-L-leucyl-L-methionine

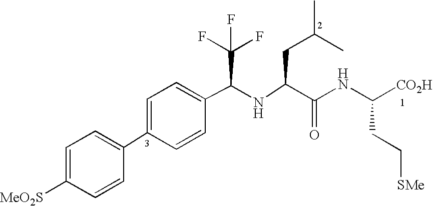

### Step 1: Methyl N-{(1S)-2,2,2-trifluoro-1-[4′-(methylsulfonyl)biphenyl-4-yl]ethyl}-L-leucyl-L-methioninate

Using the procedure described for Example 15, Step 4, where (3S)-3-amino-5-(methylthio)pentan-2-one, hydrochloride was substituted for methyl L-methionate hydrochloride, the title compound was obtained and was crystallized from EtOAc and hexane (1:2) to give a white solid. MS (+ESI): 589.3 [M+1]⁺.

### Step 2: N-{(1S)-2,2,2-trifluoro-1-[4′-(methylsulfonyl)biphenyl-4-yl]ethyl}-L-leucyl-L-methionine

To an ice-cold solution of methyl N-{(1S)-2,2,2-trifluoro-1-[4′-(methylsulfonyl)biphenyl-4-yl]ethyl}-L-leucyl-L-methioninate from Step 1 (100 mg, 0.17 mmol) in THF (2 mL) and MeOH (0.5 mL) was added 1.0N LiOH dropwise (0.25 mL, 0.25 mmol) and the resulting solution was stirred at room temperature for 18 hours. The solution was partitioned between EtOAc and water+1N HCl (1 mL). The organic layer was dried over Na₂SO₄, filtered and concentrated. The crude product was purified by chromatography using EtOH, EtOAc and AcOH as eluant to give the title compound as a white foam which was crystallized from EtOAc and hexane (6 mL, 1:2) to give a white solid.

MS (+ESI): 575.0 [M+1]⁺.

### EXAMPLE 18

### Synthesis of N¹-[(1S)-1-cyano-3-(methylthio)propyl]-N²-{(1S)-2,2,2-trifluoro-1-[4′-(methylsulfonyl)biphenyl-4-yl]ethyl}-L-leucinamide

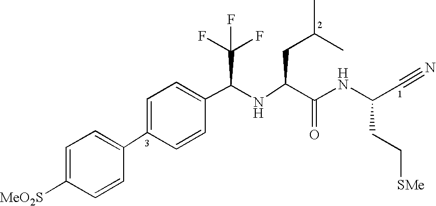

A solution of N¹-{(1S)-2,2,2-trifluoro-1-[4′-methylsulfonyl)biphenyl-4-yl]ethyl}-L-leucyl-L-methioninamide from Example 16 (350 mg, 0.6 mmol) and pyridine (85 μL, 1.05 mmol) in 1,4-dioxane was cooled to 10° C. Then, trifluoroacetic anhydride was added dropwise (65 μL, 0.46 mmol) and the reaction mixture was stirred at room temperature for 1 hour. The reaction mixture was partitioned between EtOAc and water. The organic layer was dried over Na₂SO₄, filtered and concentrated. The crude product was purified by chromatography using EtOAc and hexane as eluant to give the title compound as a colorless gum.

MS (+ESI): 556.3 [M+1]⁺.

### EXAMPLE 19

### Synthesis of N-{(1S)-2,2,2-trifluoro-1-[4′-(methylsulfonyl)biphenyl-4-yl]ethyl}-L-leucyl-N¹-methyl-L-methioninamide

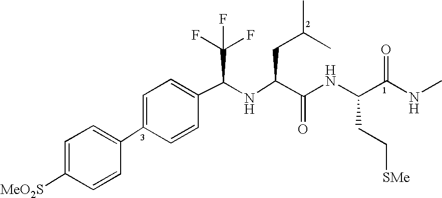

A suspension of N-{(1S)-2,2,2-trifluoro-1-[4′-(methylsulfonyl)biphenyl4-yl]ethyl)-L-leucyl-L-methionine from Example 17 (100 mg, 0.17 mmol), methylamine hydrochloride (35 mg, 0.52 mmol) and O-(7-azabenzotriazol-1-yl)-N,N,N′,N′-tetramethyluronium hexafluorophosphate (150 mg, 0.39 mmol) in DMF (1 mL) was cooled to 10° C. and then triethylamine (110 μL, 0.79 mmol) was added slowly. The reaction mixture was stirred at room temperature for 18 hours. The resulting mixture was partitioned between EtOAc and half-saturated aqueous NaHCO₃. The organic layer was dried over Na₂SO₄, filtered and concentrated. The crude product was purified by chromatography using EtOAc and hexane as eluant to give the title compound as a white foam which was crystallized from EtOAc and hexane to give a white solid.

MS (+ESI): 588.1 [M+1]⁺.

### EXAMPLE 20

### Synthesis of (2S)-2-{1(1S)-1-(4-bromophenyl)-2,2,2-trifluoroethyl]amino}-4,4-dichloro-N-(1-cyanocyclopropyl)butanamide

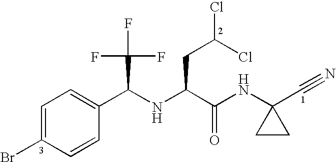

### Step 1 Ethyl (2S)-2-amino-4,4-dichlorobutanoate

To ice cold ethanol, 25 mL was added dropwise acetyl chloride (2.0 mL, 28 mmol). (S)-2-Amino-4,4-dichlorobutanoic acid (1.0 g, 5.8 mmol) [prepared according to Chem.—Ztg 114, 249-251 (1990) and Synthesis 1996, 1419] was then added in one portion. The mixture was refluxed for 18 h, concentrated and the residue partitioned between saturated NaHCO₃ solution and dichloromethane. The organics were separated dried (Na₂SO₄), filtered and concentrated to give an oily residue which was purified through a silica gel plug, eluting with 30% ethyl acetate in hexanes to give pure title compound.

### Step 2 Ethyl (2S)-2-{[(1S)-1-(4-bromophenyl)-2,2,2-trifluoroethyl]amino}-4,4-dichlorobutanoate

Ethyl (2S) -2-amino-4,4-dichlorobutanoate (298 mg, 1.49 mmol), (1S)-1-(4-bromophenyl)-2,2,2-trifluoroethyl trifluoroacetate (862 mg, 2.2 mmol) [prepared according to J. Am. Chem. Soc. 1983, 105, 2343-2350] and diisopropylethylamine (284 mg, 2.2 mmol) were heated neat at 60° C. and under N₂ atmosphere for 6 h. The mixture was partitioned between NaHCO₃ solution and ethyl acetate. The organic layer was separated, dried (Na₂SO₄), filtered and concentrated. Purification by ISCO column chromatography (gradient 2% to 15% ethyl acetate/hexanes) provided the title compound as a 87:13 mixture of diastereomers.

MS (+APCI): 438.8 [M+1] and 440.8 [M+3].

### Step 3 (2S)-2-{[(1S)-1-(4-Bromophenyl)-2,2,2-trifluoroethyl]amino}-4,4-dichlorobutanoic acid

To a solution of ethyl (2S)-2-{[(1S)-1-(4-bromophenyl)-2,2,2-trifluoroethyl]amino}-4,4-dichlorobutanoate (325 mg, 0.74 mmol) in THF (5 mL) was added potassium trimethylsilanolate (178 mg, 1.39 mmol). The mixture was stirred for 1.5 h at room temperature and then concentrated. The residue was partitioned between ethyl acetate and 1N HCl. The organic layer was separated, dried (Na₂SO₄), filtered and concentrated to obtain the title compound as an oil which was used as such in the next step.

MS (−APCI): 407.9 [M−1].

### Step 4 (2S)-2-{[(1S)-1-(4-Bromophenyl)-2,2,2-trifluoroethyl]amino}-4,4-dichloro-N-(1-cyanocyclopropyl)butanamide

A mixture of (2S)-2-{[(1S)-1-(4-bromophenyl)-2,2,2-trifluoroethyl]amino}-4,4-dichlorobutanoic acid (275 mg, 0.67 mmol), 1-amino-1-cyclopropane carbonitrile hydrochloride (159 mg, 1.34 mmol) and HATU coupling reagent (305 mg, 0.8 mmol) was dissolved in DMP (4 mL). Triethylamine (0.3 mL, 2.1 mmol) was added and the mixture stirred overnight and then poured into NaHCO₃ solution and ethyl acetate. The organic layer was separated, washed with brine, 1N HCl and brine again. The organic layer was separated, dried (Na₂SO₄), filtered and concentrated to give 393 mg of an oil which was purified by column chromatography eluting with 6:3:1 toluene: ethyl acetate:dichloromethane. Swishing the pure product with diethyl ether gave the title compound as a white solid and as a 85:15 mixture of diastereomers.

MS (−APCI): 471.9 [M−1].

¹H NMR (500 MHz, DMSO-d₆) “major isomer”, δ 8.95 (s, 1H), 7.61 (d, 2H, 7.38 (d, 2H), 6.19 (dd, 1H), 4.38-4.25 (m, 1H), 3.45-3.38 (bs, 2H), 2.5-2.3 (m, 2H), 1.43-1.32 (m, 2H), 1.02-0.95 (m, 1H), 0.75-0.68 (m, 1H).

¹H NMR (500 MHz, DMSO-d₆) “minor isomer”, δ 8.89 (s, 1H), 7.65-7.61 (m, 1H), 7.43 (d, 1H), 6.23-6.19 (m, 1H), 4.38-4.25 (m, 1H), 3.45-3.38 (bs, 2H), 2.5-2.3 (m, 2H), 1.43-1.32 (m, 2H), 1.02-0.95 (m, 1H), 0.75-0.68 (m, 1H).

### EXAMPLE 21

### N¹-(1-cyanocyclopropyl)-N²-{(1S)-2,2-difluoro-1-[4-(3-methyl-2-thienyl)phenyl]ethyl }-L-leucinamide

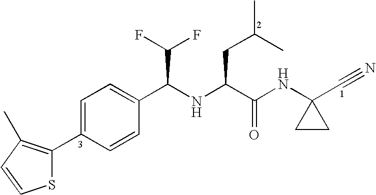

### Step 1: Preparation of (2S)-1-{[tert-butyl(dimethyl)silyl]oxy}-N-[(1Z)-2,2-difluoroethylidene]-4-methylpentan-2-amine

A mixture of (2S)-1-{[tert-butyl(dimethyl)silyl]oxy}-4-methylpentan-2-amine (Example 4, Step 1, 8.5 g, 36.8 mmol) and difluoroacetaldehyde ethyl hemiacetal (5.0 g, 39.7 mmol) in benzene was refluxed with a Dean-stark trap overnight. Solvent was removed in vacuo. The residue was passed through a short silica column and eluted with hexanes: EtOAc (10:1) to give the title compound as a pale yellow oil.

¹H NMR (CD₃COCD₃) δ 7.72(m, 1H), 6.12(dt, 1H), 3.70(dd, 1H), 3.54(dd, 1H), 3.36(m, 1H), 1.48(m, 2H), 1.32(m, 1H), 0.95-0.78(m, 15H), 0.06(s, 3H), 0.02(s, 3H).

### Step 2: Preparation of (2S)-2-{[(1S)-1-(4-bromophenyl)-2,2-difluoroethyl]amino}-4-methylpentan-1-ol

n-BuLi (2.5 M in hexanes, 1.43 mL) was added to a −70° C. THF (8.5 mL) solution of 1,4-dibromobenzene (884 mg) and the mixture was stirred for 15 minutes. A THF (8.5 mL) solution of (2S)-1-{[tert-butyl(dimethyl)siyl]oxy}-4-methyl-N-[(1E)-2,2-difluoroethylidene]pentan-2-amine (1.0 g) was then added dropwise and the mixture was stirred for 1.5 hours. It was then poured slowly into an icy saturated solution of ammonium chloride under vigorous stirring. It was extracted with 3 portions of ethyl acetate. The combined organic layers were washed with brine, dried with magnesium sulfate and the solvent was removed in vacuo to yield a residue, which was purified on SiO₂ using a gradient of hexanes and ethyl acetate (90:10 to 75:25) as eluent to yield the title compound. The compound (200 mg) from above was dissolved in CH₃CN (4 mL) and the solution was cooled to 0° C. HF-pyridine (40 □M) was added dropwise and the mixture was reacted for 16 hours. It was poured into a saturated solution of sodium bicarbonate, ethyl acetate was added and it was vigorously shaken. The organic layer was separated and the aqueous further extracted with ethyl acetate (2×50 mL). The combined organic layers were washed with brine, dried with magnesium sulfate and the solvent was removed in vacuo to yield a residue which was purified on SiO₂ using a gradient of hexanes and ethyl acetate (80:20 to 60:40) as eluent to yield the title compound.

¹H NMR (CD₃COCD₃) δ 7.6(2H, d), 7.45(2H, d), 6.0 (1H, dt), 4.25(1H, m), 3.65 (1H, t), 3.5-3.55(1H, m), 3.3-3.35(1H, m), 2.55-2.65(1H, m), 2.15-2.25(1H, m), 1.6-1.7(1H, m), 1.3-1.4(1H, m), 1.2-1.3(1H, m), 0.9(3H, d), 0.8(3H, d).

### Step 3: Preparation of N-[(1S)-1-(4-bromophenyl)-2,2-difluoroethyl]-L-leucine

A suspension of H₅IO₆/CrO₃ (5.5 mL of 0.40 M in CH3CN; see Note below) was cooled to 0° C. and a solution of the alcohol from Step 2 (250 mg) in CH₃CN (3.7 mL) was added dropwise. The mixture was stirred at 0-5° C. for 3.5 hours. After this period, 2.0 mL of the oxidant were added. After 1.5 hours it was poured into Na₂HPO₄ buffer (0.4 g in 10 mL) under vigorous stirring and the mixture was extracted with diethyl ether (3×20 mL). The combined ether extracts were washed with water and brine (1:1), with dilute aqueous NaHSO₃ and brine. The organic extract was dried with magnesium sulfate, filtered and the solvent was evaporated to dryness to yield a residue that was used without further purification.

Note: The oxidizing reagent (H₅IO₆/CrO₃) was prepared as described in Tetrahedron Letters 39 (1998) 5323-5326 but using HPLC grade CH₃CN (contains 0.5% water); no water was added.

¹H NMR (CD₃COCD₃) δ 7.55(2H, d), 7.4(2H, d), 6.05(1H, dt), 3.95-4.05(1H, m), 3.45(1H, t), 2.7-3.0(broad m, NH/OH), 1.85-1.95(1H, m), 1.5(2H, t), 0.95 (3H, d), 0.9(3H, d).

### Step 4: Preparation of N²-[(1S)-1-(4-bromophenyl)-2,2-difluoroethyl]-N¹-(1-cyanocyclopropyl)-L-leucinamide

To a DMF (2 mL) solution of the acid from Step 3 (258 mg) were added O-(7-azabenzotriazol-1-yl)-N,N,N′,N′-tetramethyluronium hexafluorophosphate (337 mg), 1-aminocyclopropanecarbonitrile hydrochloride (175 mg). After 1 minute of stirring, diisopropylethylamine (0.45 mL) was added dropwise and the mixture was stirred for 16 hours. It was poured into saturated aqueous sodium bicarbonate and extracted with ethyl acetate (3×15 mL). The combined extracts were washed with brine, dried with magnesium sulfate and the solvent removed in vacuo. The residue was purified by chromatography on SiO₂ using hexanes and ethyl acetate (80:20 to 50:50).

¹H NMR (CD₃COCD₃) δ 8.05(1H, m), 7.55(2H, d), 7.4(2H, d), 6.05(1H, dt), 3.95-4.05(1H, m), 3.25-3.3(1H, m), 2.4-2.45(1H, m), 1.8-1.9 (1H, m), 1.4-1.55(2H, m), 0.95-1.1 (2H, m), 0.95(6H, t).

### Step 5: Preparation of N1-(1-cyanocyclopropyl)-N2-{(1S)-2,2-difluoro-1-[4-(4,4,5,5-tetramethyl-1,3,2-dioxaborolan-2-yl)phenyl]ethyl}-L-leucinamide

To a DMF (60 mL) solution of the arylbromide from Step 4 (5.23 g) and of bis(pinacolato)diboron (3.8 g) were added potassium acetate (3.7 g) and PdCl₂dppf (309 mg). A stream of nitrogen was passed through the suspension for 1 minute. The reaction mixture was heated at 80° C. for 16 h. It was allowed to cool to room temperature and transferred to a sep. funnel. A saturated solution of NaHCO₃ (˜120 mL) and EtOAc (100 mL) were added. Organic layer was separated and the aqueous layer was further extracted with 2 portions of EtOAc (2×100 mL). Combined organic layers were washed with brine, dried over MgSO₄ and concentrated. Crude material was purified on silica gel (80:20 to 50:50 hex/EtOAc) to yield the desired boronate.

¹H NMR (CD₃COCD₃) δ 8.15(bs, NH), 7.72(2H, d), 7.40(2H, d), 6.02(1H, dt), 3.95(1H, m), 3.25(1H, q), 2.38(1H, m), 1.72(1H, m), 1.27-1.50(16H, m), 0.85-1.05(8H, m).

### Step 6: Preparation of N¹-(1-cyanocyclopropyl)-N²-{(1S)-2,2-difluoro-1-[4-(3-methyl-2-thienyl)phenyl]ethyl}-L-leucinamide

In a sealable tube for microwave, a stream of nitrogen was passed through a suspension made of the aryl boronate from Step 6 (200 mg), 2-bromo-3-methylthiphene (115 mg), 2 M Na₂CO₃ (0.65 mL), DMF (4.3 mL) and PdCl₂dppf (11 mg) for 1 minute. The mixture was then heated in microwave (SmithCreator) for 500 seconds (fixed hold time: OFF) at 120° C. (absorption level: high). It was cooled to room temperature, diluted with ethyl acetate (20 mL) and poured into a saturated solution of sodium bicarbonate. The ethyl acetate layer was separated and the aqueous further extracted with ethyl acetate (2×15 mL). The combined ethyl acetate extracts were washed with brine and dried with magnesium sulfate. Removal of the solvent left a residue that was purified by chromatography on SiO₂ using a gradient of hexanes and ethyl acetate (80:20 to 50:50 hex/EtOAc).

¹H NMR (CD₃COCD₃) δ 8.13(bs, NH), 7.50(4H, s), 7.37(1H, d), 6.97(1H, d), 6.05(1H, t), 4.00(1H, m), 3.30(1H, m), 2.42(1H, m), 2.32(3H, s), 1.85(1H, m), 1.40-1.53(2H, m), 1.30-1.40(2H, m), 0.85-1.03(8H, m).

### EXAMPLE 22

### Synthesis of N¹(1-cyanotolyl)-N²{(1S)-2,2,2-trifluoro-1-[4′-(methylsulfonyl)-1,1′-biphenyl-4-yl]ethyl}-L-leucinamide

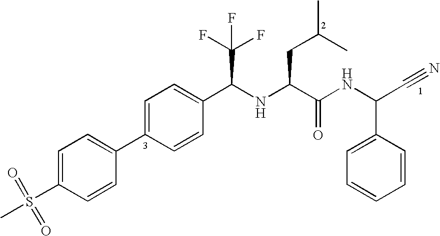

To a mixture of N-{(1S)-2,2,2-trifluoro-1-[4′-(methylsulfonyl)-1,1′-biphenyl-4-yl]ethyl}-L-leucine (Example 4, Step 6, 21 mg, 0.046 mmol) in DMF (1.4 mL) is added 2-phenylglycinonitrile hydrochloride (8.6 mg, 0.051 mmol). N-methylmorpholine (41 μL, 0.368 mmol) and N-propylphosphonic acid anhydride, cyclic trimer 50% (55 μL, 0.092 mmol) were added sequentially and the mixture was stirred overnight. The volatiles were evaporated in a Genevac HT-4. The residue was dissolved in CH₂Cl₂ (5 mL) treated 30 min with BTMA carbonate silica gel and filtered through a SiOH SPE cartridge (500 mg). The solution was treated with Amberlyst A-21 and filtered again. The solution was concentrated to give a 1:1 mixture of epimers.

MS (−ESI): 556.0 [M−1]⁻

### EXAMPLE 23

### Synthesis of N¹(cyanocyclopropyl)-N²{(1S)-2,2-difluoro-1-[2′,4′-difluoro-1,1′-biphenyl-4-yl]ethyl}-L-leucinamide

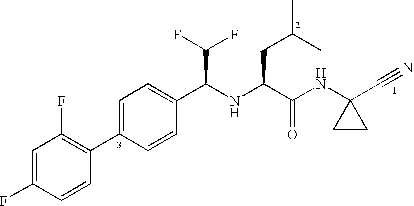

Using the procedure described for Example 21, where 2-bromo-3-methylthiophene in Step 6 was replaced by 1-bromo-2,4-difluorobenzene, the title compound was obtained as a white solid.

MS (+API): 448.1 [M+1]⁺.

### EXAMPLE 24

### Synthesis of benzyl 3-oxo4-[(N-{(1S)-2,2,2-trifluoro-1-[4′-(methylsulfonyl)biphenyl-yl]ethyl}-L-leucyl)amino]azepane-1-carboxylate

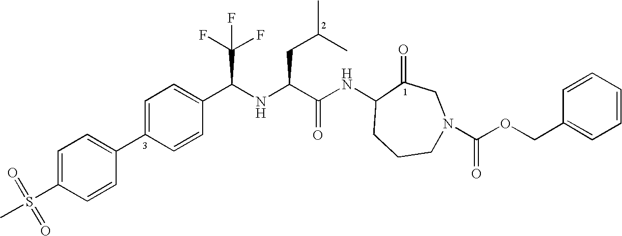

### Step 1: Benzyl 3-hydroxy-4-(N-{(1S)-2,2,2-trifluoro-1-[4′-(methylsulfonyl)biphenyl-4-yl]ethyl}-L-leucyl)amino]azepane-1-carboxylate

To a 0° C. solution of N-{(1S)-2,2,2-trifluoro-1-[4′-(methylsulfonyl)-1,1′-biphenyl-4-yl]ethyl}-L-leucine (example 4, Step 6, 605 mg, 1.37 mmol), benzyl 4-amino-3-hydroxyazepane-1-carboxylate (WO 0134565, J. Med. Chem. 44, 1380, 2001, 326 mg, 1.23 mmol), and PyBOP (724 mg, 1.39 mmol) in 10 mL of DMF was added triethylamine (0.45 mL, 3.2 mmol). The mixture was stirred 1 h, warned to room temperature for 1 h, then partitioned between NaHCO₃ and ether. The organic phase was washed with pH 3.5 phosphate buffer, then brine, and dried over MgSO₄. Purification by silica gel chromatography (gradient 65% ethyl acetate:hexanes to 100% ethyl acetate) provided the title compound as a mixture of isomers.

### Step 2: Benzyl 3-oxo4-[(N-{(1S)-2,2,2-trifluoro-1-[4′-(methylsulfonyl)biphenyl-4-yl]ethyl}-L-leucyl)amino]5 azepane-1-carboxylate

To a 0° C. solution of benzyl 3-hydroxy-4-[(N-{(1S)-2,2,2-trifluoro-1-[4′-(methylsulfonyl)biphenyl-4-yl]ethyl}-L-leucyl)amino]azepane-1-carboxylate (98 mg, 0.14 mmol) in dichloromethane (3 mL) was added Dess-Martin periodinane. The mixture was warmed to room temperature, stirred 15 h, then partitioned between ethyl acetate and 1M NaOH. The organic phase was washed with brine and dried over MgSO₄. Purification by silica gel chromatography (gradient 40% to 70% ethyl acetate:hexanes) provided the title compound as a mixture of diastereomers.

MS (+ESI): 688.4 [M+1]⁺

### EXAMPLE 25

### Synthesis of N¹-[3-oxo-1-(pyridin-2-ylsulfonyl)azepan-4-yl]-N²-(1S)-2,2,2-trifluoro-1-[4′-(methylsulfonyl)biphenyl-4-yl]ethyl}-L-leucinamide

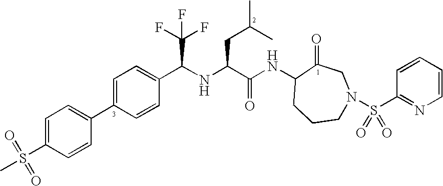

### Step 1: N¹-(3-hydroxyazepan-4yl)-N²-{(1)-2,2,2-trifluoro-1-[4′-(methylsulfonyl)biphenyl-4-yl]ethyl}-L-leucinamide

A mixture of benzyl 3-hydroxy4-[(N-{(1S)-2,2,2-trifluoro-1-[4′-(methylsulfonyl)biphenyl-4-yl]ethyl}-L-leucyl)amino]azepane-1-carboxylate (Example 24, Step 1, 710 mg, 1.03 mmol) and 10% Pd/C (490 mg) in 2:1 EtOH:EtOAc (80 mL) was flushed with hydrogen and stirred under a hydrogen balloon for 2 h. The reaction mixture was filtered through celite and concentrated to give the title compound.

### Step 2: N¹-[3-hydroxy-1-(pyridin-2-ylsulfonyl)azepan-4-yl]-N²-{(1S)-2,2,2-trifluoro-1-[4′-(methylsulfonyl)biphenyl-4-yl]ethyl}-L-leucinamide

To a 0° C. solution of N¹-(3-hydroxyazepan-4-yl)-N²-{(1S)-2,2,2-trifluoro-1-[4′-(methylsulfonyl)biphenyl-4-yl]ethyl}-L-leucinamide (567 mg, 1.02 mmol) in dichloromethane (10 mL) was added triethylamine (0.25 mL, 1.8 mmol) and 2-pyridinesulfonyl chloride (204 mg, 1.15 mmol). The mixture was warmed to room temperature for 1 h, then partitioned between dichloromethane and NaHCO₃. The organic phase was washed with brine, filtered through cotton and concentrated. Purification by silica gel chromatography (gradient 70% ethyl acetate:hexanes to 100% ethyl acetate) provided the title compound as a mixture of isomers.

### Step 3: N¹-[3-oxo-1-(pyridin-2-ylsulfonyl)azepan-4-yl]-N²-{(1S)-2,2,2-trifluoro-1-[4′-(methylsulfonyl)biphenyl-4-yl]ethyl}-L-leucinamide

To a room temperature solution of N¹-[3-hydroxy-1-(pyridin-2-ylsulfonyl)azepan-4-yl]-N²-{(1S)-2,2,2-trifluoro-1-[4′-(methylsulfonyl)biphenyl-4-yl]ethyl}-L-leucinamide (460 mg, 0.73 mmol) in dichloromethane (15 mL) was added Dess-Martin periodinane (420 mg, 1.0 mmol). The mixture was stirred 1 h, then diluted with dichloromethane, washed with 1M NaOH, then brine. The organic phase was filtered through cotton and concentrated. Purification by silica gel chromatography (gradient 60% ethyl acetate:hexanes to 100% ethyl acetate) provided the title compound as a mixture of diastereomers.

MS (+ESI): 695.3 [M+1]⁺

### EXAMPLE 26

### Synthesis of N²-[(4-bromophenyl)(4-methoxyphenyl)methyl]-N′-(cyanomethyl)-L-leucinamide

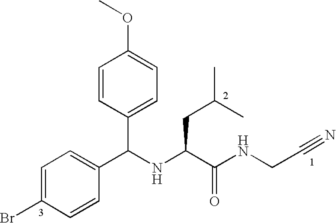

### Step 1: (4-bromophenyl)(4-methoxyphenyl)methanol

A solution of 1,4-dibromobenzene (9.1 g, 38 mmoles) in TBF (80 mL) was cooled to −78° C. and n-butyllithium (16 mL, 2.5M in hexanes) was added dropwise. After 15 minutes, p-anisaldehyde (5 g, 37 mmoles in 4.5 mL of THF) was added dropwise. After another 20 minutes, the reaction mixture was quenched with methanol (5 mL) and saturated aqueous ammonium chloride (100 mL). The reaction mixture was extracted with three 100-mL portions of ethyl acetate and the combined organic layers were washed with brine (50 mL). The organic layer was dried over magnesium sulfate and concentrated under reduced pressure to give the title compound which was used directly in the next step.

### Step 2: Methyl N-[(4-bromophenyl)(4-methoxyphenyl)methyl]-L-leucinate

(4-Bromophenyl)(4-methoxyphenyl)methanol (1.15 g, 3.9 mmoles) was dissolved in dichloromethane (4 mL) along with tetrabutylammonium bromide (130 mg, 0.4 mmoles). 48% aqueous hydrobromic acid (3.3 mL) was then added and the reaction mixture stirred vigourously for 40 h. The products were partitioned between water (20 mL) and dichloromethane (30 mL) and dried over magnesium sulfate. The organic layer was concentrated to approximately 10 mL and L-leucine methyl ester (free base) was added as a solution in dichloromethane (20 mL) followed by triethylamine (3 mL). The reaction mixture was stirred at 35° C. for 20 minutes (a white precipitate appeared). The reaction was taken-up in ether (50 mL) and water (30 mL). The phases were separated and the organic layer washed with saturated aqueous ammonium chloride solution (30 mL) and brine (30 mL). After drying over magnesium sulfate and concentrating under reduced pressure, methyl N-[(4-bromophenyl)(4-methoxyphenyl)methyl]-L-leucinate was obtained and purified on silica gel using 10% ethyl acetate, 90% hexanes.

### Step 3: N-[(4-bromophenyl)(4-methoxyphenyl)methyl]-L-leucine

To a room temperature solution of methyl N-[(4-bromophenyl)(4-methoxyphenyl)methyl]-L-leucinate (1.25 g, 3 mmol) in 60 mL of approximately 2:1:1 THF/MeOH/Water was added lithium hydroxide monohydrate (250 mg, 6 mmoles). The mixture was stirred overnight and concentrated. The residue was partitioned between dichloromethane (50 mL) and pH 3.5 phosphate buffer (50 mL). The aqueous phase was separated and washed with two 50-mL portions of dichloromethane. The organic phase was dried over sodium sulfate and concentrated under reduced pressure to yield a solid that was triturated in a minimum of cold dichloromethane to give N-[(4-bromophenyl)(4-methoxyphenyl)methyl]-L-leucine.

### Step 4: N²-[(4-bromophenyl)(4-methoxyphenyl)methyl]-N¹-(cyanomethyl)-L-leucinamide

A mixture of N-[(4-bromophenyl)(4-methoxyphenyl)methyl]-L-leucine (149 mg, 0.37 mmol) and aminoacetonitrile hydrochloride (87 mg, 0.73 mmol) was dissolved in 5 mL of dimethylformamide. HATU (153 mg, 0.403 mmol) was added in one portion followed by triethylamine (0.18 mL, 1.31 mmol). The mixture was stirred overnight, then poured into pH 4 phosphate buffer (40 mL) and extracted with ethyl acetate (50 mL). The organic phase was washed with three 50-mL portions of water, dried over sodium sulfate and concentrated under reduced pressure. Purification over silica gel (30% ethyl acetate/hexanes) provided N²-[(4-bromophenyl)(4-methoxyphenyl)methyl]-N′-(cyanomethyl)-L-leucinamide.

MS (+ESI): 443.9 [M+1].

### Cathespin Binding

Each of Examples 1 through 26 was submitted to the following procedure: within the context of a computer-generated model, the compound was energy-minimized in the active site of Cathepsin K. The computer-generated model of Cathepsin K was based on the crystal structures of the Protein Databank entry 1 MEM, to which hydrogens had been added, but no energy minimization of non-hydrogens had been performed at the start of the energy minimization (therefore protein sidechains retained the geometry of the Xray crystal structure). The binding orientation of the compound was determined based on the following assumptions: 1) that a covalent bond is formed in Si between the electrophilic carbon labeled “1” in the compound. In conjunction with the chemical formula for the invention, this determines the molecular fragments corresponding to R₁ and R₂; 2) for the fragment of the compound corresponding to the formula of the invention, the amine hydrogen forms a hydrogen bond with the oxygen of Gly66, therefore having a distance of less than 4 Å between these two atoms; 3) The fragments of the compound corresponding to R₂ and R₃ were placed in the cathepsin subsites S₂ and S₃, respectively, according to the labeled carbon atoms on the chemical structure for the compound. For example, the carbon labeled “2” in the structure was located in S₂ and the carbon labeled “3” was located in S₃, such that the distances to cathepsin Cα's in Table 1 were within an angstrom of those given in Table 1. Although these placements were done manually and approximately, they were placed intentionally to lead to favorable interactions. For compounds synthesized as racemic mixtures, the enantiomer corresponding to the chemical formula of the invention was used for the calculation. The energy minimization was carried out using the software MacroModel with the MMFFs force field. All atoms of the compound were allowed to move, but for Cathepsin K only protein sidechains having an atom within 6 Å of the compound were allowed to move; in this case the whole sidechain could move. Default parameters for the energy minimization were chosen, with a continuum solvent option corresponding to water. The results of the energy minimization for the compound was favorable: there were no bad steric interactions between the ligand and the active site of Cathepsin K, and favorable interactions between the active site and the ligand (lipophilic interactions and hydrogen bonding) are confirmed by the distances given in Table 1. Table 1 gives distances taken from the energy-minimized ligand-enzyme complexes formed by the compounds and Cathepsin K as described above. The column labeled “Gly66 Hbond” gives the distance of the hydrogen bond formed between the amine hydrogen of the compound and the oxygen of Gly66. The three columns under R2 in Table 1 are headed by a Cα label corresponding to a Cα in the cathepsin; these columns give the distance between the indicated Cα in the cathepsin and the carbon labeled “2” in the structure for that compound. Similarly, the two columns under “R3” in Table 1 give the distance between the indicated Cα in the cathepsin and the carbon labeled “3” in the structure for that compound. The covalent bond made between cysteine sulfur of residue 25 in the cathepsin and the electrophilic carbon labeled “1” in the structure for that compound ensured distance of <5 Å of the carbon labeled “1” in the structure for that compound. Thus Examples 1 through 26 meet the distance criteria described herein.

TABLE 1
Distances (in Å) from atoms in Cathepsin K to atoms in Examples 1 to 26.
See text.

| | | R2 | | | R3 | |
|---|---|---|---|---|---|---|
| Example | Gly66 Hbond | Cα₂₆ | Cα₆₈ | Cα₁₃₄ | Cα₆₆ | Cα₆₀ |
| 1 | 2.4 | 6.4 | 7.7 | 5.5 | 4.5 | 6.1 |
| 2 | 2.5 | 6.4 | 7.7 | 5.5 | 4.4 | 5.9 |
| 3 | 2.5 | 6.5 | 7.7 | 5.5 | 4.8 | 6.3 |
| 4 | 2.6 | 6.4 | 7.7 | 5.6 | 4.8 | 6.3 |
| 5 | 2.6 | 6.4 | 7.7 | 5.4 | 4.8 | 6.3 |
| 6 | 2.5 | 6.4 | 7.7 | 5.5 | 4.7 | 6.3 |
| 7 | 2.5 | 6.4 | 7.7 | 5.5 | 4.6 | 6.1 |
| 8 | 2.5 | 6.5 | 7.6 | 5.4 | 4.7 | 6.3 |
| 9 | 2.4 | 6.5 | 7.8 | 5.3 | 4.6 | 6.2 |
| 10 | 2.5 | 6.8 | 8.0 | 5.4 | 4.6 | 6.1 |
| 11 | 2.6 | 6.6 | 7.7 | 5.4 | 4.8 | 6.3 |
| 12 | 2.6 | 6.2 | 7.7 | 5.6 | 4.8 | 6.5 |
| 13 | 2.7 | 6.7 | 7.9 | 5.6 | 5.0 | 6.5 |
| 14 | 2.5 | 6.5 | 7.7 | 5.5 | 4.5 | 6.0 |
| 15 | 2.5 | 6.5 | 7.7 | 5.4 | 4.7 | 6.3 |
| 16 | 2.4 | 6.5 | 7.5 | 5.4 | 4.7 | 6.2 |
| 17 | 2.4 | 6.6 | 7.5 | 5.4 | 4.7 | 6.2 |
| 18 | 2.4 | 6.4 | 7.5 | 5.4 | 4.7 | 6.2 |
| 19 | 2.5 | 6.7 | 7.5 | 5.4 | 4.8 | 6.2 |
| 20 | 2.3 | 6.4 | 7.5 | 5.5 | 4.5 | 6.1 |
| 21 | 2.4 | 6.4 | 7.6 | 5.4 | 4.6 | 6.2 |
| 22 | 2.5 | 6.5 | 7.6 | 5.5 | 4.7 | 6.2 |
| 23 | 2.4 | 6.4 | 7.6 | 5.4 | 4.7 | 6.3 |
| 24 | 2.0 | 6.7 | 7.3 | 5.4 | 4.6 | 6.0 |
| 25 | 2.4 | 6.5 | 7.6 | 5.4 | 4.6 | 6.2 |
| 26 | 2.4 | 6.4 | 7.7 | 5.5 | 4.8 | 6.4 |

### Purified Enzyme Assays

The compounds disclosed in the present application exhibited activity in the following assays. In addition, the compounds disclosed in the present application have an enhanced pharmacological profile relative to previously disclosed compounds.

### Cathepsin K Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 μL of human cathepsin K (0.4 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 um) for 10 minutes. Percent of inhibition were calculated by fitting experimental values to standard mathematical model for dose response curve.

### Cathepsin L Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 μL of human cathepsin L (0.5 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Cathepsin B Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 μL of human cathepsin B (4.0 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Cathepsin S Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; DTT, 2.5 mM and 10% DMSO) and 25 μL of human cathepsin S (20 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

What is claimed is:

1. A compound having the chemical formula: 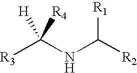
and having no more than 70 nonhydrogen atoms each independently selected from C, O, N, S, P, F, Cl, Br or I;
wherein R₄ is a non-hydrogen electron-withdrawing substituent such that, together with R₁, R₂ and R₃, the basicity of the nitrogen is lowered to less than a pKa of 6;
wherein a molecule of the compound is interacting with a cathepsin such that the CH—NH region in the chemical formula is interacting favorably with the cathepsin between S2 and S3, R₁ interacts favorably with S1 but not S3 of the active site of the cathepsin active site, R₂ interacts favorably with S2 but not S3 of the cathepsin active site, and R₃ interacts favorably with S3 but not S2 or S1 of the cathepsin active site.

2. The compound of claim 1 wherein the cathepsin is selected from cathepsin B, F, H, K, L, L₂, O, S, W or Z.

3. The compound of claim 2 wherein the cathepsin is selected from cathepsin K, L, S or B.

4. The compound of claim 1, wherein R₄ is not interacting favorably with subsites S₂, S₃ and S₁, respectively, of a cathepsin active site.

5. The compound of claim 1, wherein R₂ has at least one carbon or sulfur atom which simultaneously fulfills the following three distance critieria: it is within 7 Å of Cα₂₆, and it is within 8.5 Å of Cα₆₈ and it is within 7 Å of Cα₁₃₄ of a cathepsin.

6. The compound of claim 1, wherein R₃ has at least one carbon or sulfur atom which simultaneously fulfills the following two distance critieria: it is within 5.5 Å of Cα₆₆, and it is within 7 Å of Cα₆₀ of a cathepsin.

7. The compound of claim 1, wherein the nitrogen has a pKa of less than 6 and makes a hydrogen bond with the cathepsin amide carbonyl of glycine 66 of a cathepsin.

8. The compound of claim 1, wherein R₂ comprises nonpolar regions.

9. The compound of claim 1, wherein R₂ comprises lipophilic regions.

10. The compound of claim 1, wherein R₃ comprises nonpolar regions.

11. The compound of claim 1, wherein R₃ comprises lipophilic regions.

12. The compound of claim 1, wherein the pKa of the nitrogen of the secondary amine shown in claim 1 is <5 in an aqueous medium.

13. The compound of claim 1, wherein R₄ is a group selected from —CF₃, —CHF₂, —CH₂F, —CF₂R₅, and —CHFR₅, wherein R₅ is C₁₋₆ alkyl, aryl or heteroaryl optionally substituted with 1 to 4 substituents selected from halo, C₁₋₃ alkyl, C₁₋₃ alkoxy, hydroxy, hydroxyalkyl, keto, cyano, heterocyclyl, C₃₋₈ cycloalkyl, SOₘC₁₋₃ alkyl, NH₂, NO₂ or O(C═O)C₁₋₃ alkyl; and m is an integer from zero to two.

14. The compound of claim 1, wherein the R₁ comprises a region that stably fits into subsite SI of a cathepsin active site, having at least one carbon atom within 5 Å of Cα₂₅ of a cathepsin.

15. The compound of claim 14 wherein the cathepsin is selected from cathepsin B, F, H, K, L, L2, O, S, W or Z.

16. The compound of claim 14, wherein the compound forms a covalent bond with the sulfur of cysteine 25 of a cathepsin.

17. The compound of claim 14, wherein R₁ is non-immunogenic.

18. The compound of claim 14, wherein the compound binds to the active site of a cathepsin with an IC₅₀ of less than 10 micromolar in a purified enzyme assay.

19. The compound of claim 14, wherein a covalent bond is made to an electrophilic carbonyl carbon of the compound.

20. The compound of claim 1, wherein no covalent bond is formed between the compound and a cathepsin.

21. A pharmaceutical composition comprising a compound as defined in any one of claims 1 to 20, or a pharmaceutically acceptable salt thereof, in association with a pharmaceutically acceptable carrier.

22. Use of a compound as defined in any one of claims 1 to 20, or a pharmaceutically acceptable salt thereof, in the manufacture of a medicament for inhibiting cathepsin activity, or treating or preventing cathepsin dependent conditions in a mammal, or inhibiting bone loss or reducing bone loss in a mammal, or treating or preventing osteoporosis in a mammal, or treating or preventing rheumatoid arthritis condition in a mammal, or treating or preventing progression of osteoarthritis in a mammal, or treating cancer in a mammal.

23. A compound as defined in any one of claims 1 to 20, or a pharmaceutically acceptable salt thereof for use in a medicament therapy.

24. A method of treating or preventing cathepsin dependent conditions in a mammal, or inhibiting bone loss or reducing bone loss in a mammal, or treating or preventing osteoporosis in a mammal, or treating or preventing rheumatoid arthritis condition in a mammal, or treating or preventing progression of osteoarthritis in a mammal, or treating cancer in a mammal comprising administering to a mammal a therapeutically effective amount of a compound as defined in any one of claims 1 to 20, or a pharmaceutically acceptable salt thereof.


SEQUENCE LISTING (13 sequences)

SEQ ID NO: 1 - protein, 215 aa, HOMO SAPIENS
APDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 2 - protein, 254 aa, HOMO SAPIENS
LPASFDAREQWPQCPTIKEIRDQGSCGSCMAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTD

SEQ ID NO: 3 - protein, 214 aa, HOMO SAPIENS
APPEWDWRSKGAVTKVKDQGMCGSCWAFSVTGNVEGQWFLNQGTLLSLSEQELLDCDKMDKACMGGLPSNAYSAIKNLGGLETEDDYSYQGHMQSCNFSAEKAKVYINDSVELSQNEQKLAAWLAKRGPISVAINAFGMQFYRHGISRPLRPLCSPWLIDHAVLLVGYGNRSDVPFWAIKNSWGTDWGEKGYYYLHRGSGACGVNTMASSAVVD

SEQ ID NO: 4 - protein, 220 aa, HOMO SAPIENS
YPPSVDWRKKGNFVSPVKNQGACGSCWTFSTTGALESAIAIATGKMLSLAEQQLVDCAQDFNNHGCQGGLPSQAFEYILYNKGIMGEDTYPYQGKDGYCKFQPGKAIGFVKDVANITIYDEEAMVEAVALYNPVSFAFEVTQDFMMYRTGIYSSTSCHKTPDKVNHAVLAVGYGEKNGIPYWIVKNSWGPQWGMNGYFLIERGKNMCGLAACASYPIPLV

SEQ ID NO: 5 - protein, 220 aa, HOMO SAPIENS
APRSVDWREKGYVTPVKNQGQCGSCWAFSATGALEGQMFRKTGRLISLSEQNLVDCSGPQGNEGCNGGLMDYAFQYVQDNGGLDSEESYPYEATEESCKYNPKYSVANDTGFVDIPKQEKALMKAVATVGPISVAIDAGHESFLFYKEGIYFEPDCSSEDMDHGVLVVGYGFESTESDNNKYWLVKNSWGEEWGMGGYVKMAKDRRNHCGIASAASYPTV

SEQ ID NO: 6 - protein, 214 aa, HOMO SAPIENS
LPLRFDWRDKQVVTQVRNQQMCGGCWAFSVVGAVESAYAIKGKPLEDLSVQQVIDCSYNNYGCNGGSTLNALNWLNKMQVKLVKDSEYPFKAQNGLCHYFSGSHSGFSIKGYSAYDFSDQEDEMAKALLTFGPLVVIVDAVSWQDYLGGIIQHHCSSGEANHAVLITGFDKTGSTPYWIVRNSWGSSWGVDGYAHVKMGSNVCGIADSVSSIFV

SEQ ID NO: 7 - protein, 217 aa, HOMO SAPIENS
LPDSVDWREKGCVTEVKYQGSCGACWAFSAVGALEAQLKLKTGKLVTLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 8 - protein, 249 aa, HOMO SAPIENS
VPFSCDWRKVAGAISPIKDQKNCNCCWAMAAAGNIETLWRISFWDFVDVSVHELLDCGRCGDGCHGGFVWDAFITVLNNSGLASEKDYPFQGKVRAHRCHPKKYQKVAWIQDFIMLQNNEHRIAQYLATYGPITVTINMKPLQLYRKGVIKATPTTCDPQLVDHSVLLVGFGSVKSEEGIWAETVSSQSQPQPPHPTPYWILKNSWGAQWGEKGYFRLHRGSNTCGITKFPLTARVQKPDMKPRVSCPP

SEQ ID NO: 9 - protein, 242 aa, HOMO SAPIENS
LPKSWDWRNVDGVNYASITRNQHIPQYCGSCWAHASTSAMADRINIKRKGAWPSTLLSVQNVIDCGNAGSCEGGNDLSVWDYAHQHGIPDETCNNYQAKDQECDKFNQCGTCNEFKECHAIRNYTLWRVGDYGSLSGREKMMAEIYANGPISCGIMATERLANYTGGIYAEYQDTTYINHVVSVAGWGISDGTEYWIVRNSWGEPWGERGWLRIVTSTYKDGKGARYNLAIEEHCTFGDPIV

SEQ ID NO: 10 - protein, 221 aa, HOMO SAPIENS
LPKSVDWRKKGYVTPVKNQKQCGSCWAFSATGALEGQMFRKTGKLVSLSEQNLVDCSRPQGNQGCNGGFMARAFQYVKENGGLDSEESYPYVAVDEICKYRPENSVANDTGFTVVAPGKEKALMKAVATVGPISVAMDAGHSSFQFYKSGIYFEPDCSSKNLDHGVLVVGYGFEGANSNNSKYWLVKNSWGPEWGSNGYVKIAKDKNNHCGIATAASYPNV

SEQ ID NO: 11 - protein, 237 aa, PLASMODIUM FALCIPARUM
VPEILDYREKGIVHEPKDQGLCGSCWAFASVGNIESVFAKKNKNILSFSEQEVVDCSKDNFGCDGGHPFYSFLYVLQNELCLGDEYKYKAKDDMFCLNYRCKRKVSLSSIGAVKENQLILALNEVGPLSVNVGVNNDFVAYSEGVYNGTCSEELNHSVLLVGYGQVEKTKLNYNNKIQTYNTKENSNQPDDNIIYYWIIKNSWSKKWGENGFMRLSRNKNGDNVFCGIGEEVFYPIL

SEQ ID NO: 12 - protein, 222 aa, PLASMODIUM FALCIPARUM
DRIAYDWRLHGGVTPVKDQALCGSCWAFSSVGSVESQYAIRKLFLFSEQELVDCSVKNNGCYGGYITNAFDDMIDLGGLCSQDDYPYVSNLPETCNLKRCNERYTIKSYVSIPDDKFKEALRYLGPISISIAASDDFAFYRGGFYDGECGAAPNHAVILVGYGMKDIYNEDTGRMEKFYYYIIKNSWGSDWGEGGYINLETDENGYKKTCSIGTEAYVPLLE

SEQ ID NO: 13 - protein, 224 aa, PLASMODIUM FALCIPARUM
DHAAYDWRLHSGVTPVKDQKNCGSCWAFSSIGSVESQYAIRKNKLITLSEQELVDCSFKNYGCNGGLINNAFEDMIELGGICPDGDYPYVSDAPNLCNIDRCTEKYGIKNYLSVPDNKLKEALRFLGPISISVAVSDDFAFYKEGIFDGECGDELNHAVMLVGFGMKEIVNPLTKKGEKHYYYIIKNSWGQQWGERGFINIETDESGLMRKCGLGTDAFIPLIE